SEQUENCE LISTING (43 sequences)

SEQ ID NO: 1 - DNA, 7904 bp, Human papillomavirus type 16
actacaataattcatgtataaaactaagggcgtaaccgaaatcggttgaaccgaaaccggttagtataaaagcagacattttatgcaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaatcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataatctaccatggctgatcctgcaggtaccaatggggaagagggtacgggatgtaatggatggttttatgtagaggctgtagtggaaaaaaaaacaggggatgctatatcagatgacgagaacgaaaatgacagtgatacaggtgaagatttggtagattttatagtaaatgataatgattatttaacacaggcagaaacagagacagcacatgcgttgtttactgcacaggaagcaaaacaacatagagatgcagtacaggttctaaaacgaaagtatttggtagtccacttagtgatattagtggatgtgtagacaataatattagtcctagattaaaagctatatgtatagaaaaacaaagtagagctgcaaaaaggagattatttgaaagcgaagacagcgggtatggcaatactgaagtggaaactcagcagatgttacaggtagaagggcgccatgagactgaaacaccatgtagtcagtatagtggtggaagtgggggtggttgcagtcagtacagtagtggaagtgggggagagggtgttagtgaaagacacactatatgccaaacaccacttacaaatattttaaatgtactaaaaactagtaatgcaaaggcagcaatgttagcaaaatttaaagagttatacggggtgagtttttcagaattagtaagaccatttaaaagtaataaatcaacgtgttgcgattggtgtattgctgcatttggacttacacccagtatagctgacagtataaaaacactattacaacaatattgtttatatttacacattcaaagtttagcatgttcatggggaatggttgtgttactattagtaagatataaatgtggaaaaaatagagaaacaattgaaaaattgctgtctaaactattatgtgtgtctccaatgtgtatgatgatagagcctccaaaattgcgtagtacagcagcagcattatattggtataaaacaggtatatcaaatattagtgaagtgtatggagacacgccagaatggatacaaagacaaacagtattacaacatagttttaatgattgtacatttgaattatcacagatggtacaatgggcctacgataatgacatagtagacgatagtgaaattgcatataaatatgcacaattggcagacactaatagtaatgcaagtgcctttctaaaaagtaattcacaggcaaaaattgtaaaggattgtgcaacaatgtgtagacattataaacgagcagaaaaaaaacaaatgagtatgagtcaatggataaaatatagatgtgatagggtagatgatggaggtgattggaagcaaattgttatgtttttaaggtatcaaggtgtagagtttatgtcatttttaactgcattaaaaagatttttgcaaggcatacctaaaaaaaattgcatattactatatggtgcagctaacacaggtaaatcattatttggtatgagtttaatgaaatttctgcaagggtctgtaatatgttttgtaaattctaaaagccatttttggttacaaccattagcagatgccaaaataggtatgttagatgatgctacagtgccctgttggaactacatagatgacaatttaagaaatgcattggatggaaatttagtttctatggatgtaaagcatagaccattggtacaactaaaatgccctccattattaattacatctaacattaatgctggtacagattctaggtggccttatttacataatagattggtggtgtttacatttcctaatgagtttccatttgacgaaaacggaaatccagtgtatgagcttaatgataagaactggaaatcctttttctcaaggacgtggtccagattaagtttgcacgaggacgaggacaaggaaaacgatggagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaagtggtgccaacactggctgtatcaaagaataaagcattacaagcaattgaactgcaactaacgttagaaacaatatataactcacaatatagtaatgaaaagtggacattacaagacgttagccttgaagtgtatttaactgcaccaacaggatgtataaaaaaacatggatatacagtggaagtgcagtttgatggagacatatgcaatacaatgcattatacaaactggacacatatatatatttgtgaagaagcatcagtaactgtggtagagggtcaagttgactattatggtttatattatgttcatgaaggaatacgaacatattttgtgcagtttaaagatgatgcagaaaaatatagtaaaaataaagtatgggaagttcatgcgggtggtcaggtaatattatgtcctacatctgtgtttagcagcaacgaagtatcctctcctgaaattattaggcagcacttggccaaccaccccgccgcgacccataccaaagccgtcgccttgggcaccgaagaaacacagacgactatccagcgaccaagatcagagccagacaccggaaacccctgccacaccactaagttgttgcacagagactcagtggacagtgctccaatcctcactgcatttaacagctcacacaaaggacggattaactgtaatagtaacactacacccatagtacatttaaaaggtgatgctaatactttaaaatgtttaagatatagatttaaaaagcattgtacattgtatactgcagtgtcgtctacatggcattggacaggacataatgtaaaacataaaagtgcaattgttacacttacatatgatagtgaatggcaacgtgaccaatttttgtctcaagttaaaataccaaaaactattacagtgtctactggatttatgtctatatgacaaatcttgatactgcatccacaacattactggcgtgctttttgctttgctttgtgtgcttttgtgtgtctgcctattaatacgtccgctgcttttgtctgtgtctacatacacatcattaataatattggtattactattgtggataacagcagcctctgcgtttaggtgttttattgtatatattatatttgtttatataccattatttttaatacatacacatgcacgctttttaattacataatgtatatgtacataatgtaattgttacatataattgttgtataccataacttactattttttcttttttattttcatatataatttttttttttgtttgtttgtttgttttttaataaactgttattacttaacaatgcgacacaaacgttctgcaaaacgcacaaaacgtgcatcggctacccaactttataaaacatgcaaacaggcaggtacatgtccacctgacattatacctaaggttgaaggcaaaactattgctgaacaaatattacaatatggaagtatgggtgtattttttggtgggttaggaattggaacagggtcgggtacaggcggacgcactgggtatattccattgggaacaaggcctcccacagctacagatacacttgctcctgtaagaccccctttaacagtagatcctgtgggcccttctgatccttctatagtttctttagtggaagaaactagttttattgatgctggtgcaccaacatctgtaccttccattcccccagatgtatcaggatttagtattactacttcaactgataccacacctgctatattagatattaataatactgttactactgttactacacataataatcccactttcactgacccatctgtattgcagcctccaacacctgcagaaactggagggcattttacactttcatcatccactattagtacacataattatgaagaaattcctatggatacatttattgttagcacaaaccctaacacagtaactagtagcacacccataccagggtctcgcccagtggcacgcctaggattatatagtcgcacaacacaacaggttaaagttgtagaccctgcttttgtaaccactcccactaaacttattacatatgataatcctgcatatgaaggtatagatgtggataatacattatatttttctagtaatgataatagtattaatatagctccagatcctgactttttggatatagttgctttacataggccagcattaacctctaggcgtactggcattaggtacagtagaattggtaataaacaaacactacgtactcgtagtggaaaatctataggtgctaaggtacattattattatgatttaagtactattgatcctgcagaagaaatagaattacaaactataacaccttctacatatactaccacttcacatgcagcctcacctacttctattaataatggattatatgatatttatgcagatgactttattacagatacttctacaaccccggtaccatctgtaccctctacatctttatcaggttatattcctgcaaatacaacaattccttttggtggtgcatacaatattcctttagtatcaggtcctgatatacccattaatataactgaccaagctccttcattaattcctatagttccagggtctccacaatatacaattattgctgatgcaggtgacttttatttacatcctagttattacatgttacgaaaacgacgtaaacgtttaccatattttttttcagatgtctctttggctgcctagtgaggccactgtctacttgcctcctgtcccagtatctaaggttgtaagcacggatgaatatgttgcacgcacaaacatatattatcatgcaggaacatccagactacttgcagttggacatccctattttcctattaaaaaacctaacaataacaaaatattagttcctaaagtatcaggattacaatacagggtatttagaatacatttacctgaccccaataagtttggttttcctgacacctcattttataatccagatacacagcggctggtttgggcctgtgtaggtgttgaggtaggtcgtggtcagccattaggtgtgggcattagtggccatcctttattaaataaattggatgacacagaaaatgctagtgcttatgcagcaaatgcaggtgtggataatagagaatgtatatctatggattacaaacaaacacaattgtgtttaattggttgcaaaccacctataggggaacactggggcaaaggatccccatgtaccaatgttgcagtaaatccaggtgattgtccaccattagagttaataaacacagttattcaggatggtgatatggttcatactggctttggtgctatggactttactacattacaggctaacaaaagtgaagttccactggatatttgtacatctatttgcaaatatccagattatattaaaatggtgtcagaaccatatggcgacagcttatttttttatttacgaagggaacaaatgtttgttagacatttatttaatagggctggtactgttggtgaaaatgtaccagacgatttatacattaaaggctctgggtctactgcaaatttagccagttcaaattattttcctacacctagtggttctatggttacctctgatgcccaaatattcaataaaccttattggttacaacgagcacagggccacaataatggcatttgttggggtaaccaactatttgttactgttgttgatactacacgcagtacaaatatgtcattatgtgctgccatatctacttcagaaactacatataaaaatactaactttaaggagtacctacgacatggggaggaatatgatttacagtttatttttcaactgtgcaaaataaccttaactgcagacgttatgacatacatacattctatgaattccactattttggaggactggaattttggtctacaacctcccccaggaggcacactagaagatacttataggtttgtaacccaggcaattgcttgtcaaaaacatacacctccagcacctaaagaagatgatccccttaaaaaatacactttttgggaagtaaatttaaaggaaaagttttctgcagacctagatcagtttcctttaggacgcaaatttttactacaagcaggattgaaggccaaaccaaaatttacattaggaaaacgaaaagctacacccaccacctcatctacctctacaactgctaaacgcaaaaaacgtaagctgtaagtattgtatgtatgttgaattagtgttgtttgttgtgtatatgtttgtatgtgcttgtatgtgcttgtaaatattaagttgtatgtgtgtttgtatgtatggtataataaacacgtgtgtatgtgtttttaaatgcttgtgtaactattgtgtcatgcaacataaataaacttattgtttcaacacctactaattgtgttgtggttattcattgtatataaactatatttgctacatcctgtttttgttttatatatactatattttgtagcgccaggcccattttgtagcttcaaccgaattcggttgcatgctttttggcacaaaatgtgtttttttaaatagttctatgtcagcaactatggtttaaacttgtacgtttcctgcttgccatgcgtgccaaatccctgttttcctgacctgcactgcttgccaaccattccattgttttttacactgcactatgtgcaactactgaatcactatgtacattgtgtcatataaaataaatcactatgcgccaacgccttacataccgctgttaggcacatatttttggcttgttttaactaacctaattgcatatttggcataaggtttaaacttctaaggccaactaaatgtcaccctagttcatacatgaactgtgtaaaggttagtcatacattgttcatttgtaaaactgcacatgggtgtgtgcaaaccgattttgggttacacatttacaagcaacttatataataatactaa

SEQ ID NO: 2 - DNA, 7942 bp, Human papillomavirus type 52
taaattataatcttatactagtaaaaaatagggtgtaaccgaaaacggtcagaccgaaaccggtgtatatatatagaacacagtgtagctaacgcacggccatgtttgaggatccagcaacacgaccccggaccctgcacgaattgtgtgaggtgctggaagaatcggtgcatgaaataaggctgcagtgtgtgcagtgcaaaaaagagctacaacgaagagaggtatacaagtttctatttacagatttacgaatagtatatagagacaataatccatatggcgtgtgtattatgtgcctacgctttttatctaagataagtgaatataggcattatcaatattcactgtatgggaaaacattagaagagagggtaaaaaaaccattaagtgaaataactattagatgtataatttgtcaaacgccattatgtcctgaagaaaaagaaagacatgttaatgcaaacaagcgatttcataatattatgggtcgttggacagggcgctgttcagagtgttggagaccccgacctgtgacccaagtgtaacgtcatgcgtggagacaaagcaactataaaagattatatattagatctgcaacctgaaacaactgacctacactgctatgagcaattaggtgacagctcagatgaggaggatacagatggtgtggaccggccagatggacaagcagaacaagccacaagcaattactacattgtgacatattgtcacagttgtgatagcacactacggctatgcattcatagcactgcgacggaccttcgtactctacagcaaatgctgttgggcacattacaagttgtgtgccccggctgtgcacggctataaacaaccctgcaatggaggaccctgaaggtacagagggcgaaagggagggatgtacaggctggtttgaagtagaggcaataatagaaaaacaaacaggagataacatttcagaggacgaggatgaaaatgcatatgatagtggaacagatctaatagattttatagatgattcaaatataaataatgaacaggcagaacatgaggcagcccgggcattgtttaatgcacaggaaggggaggatgatttacatgctgtgtctgcagtaaaacgaaagtttacaagcagtccggaaagtgctgggcaagatggtgtagaaaaacatggtagtccgcgtgcaaaacacatttgtgtaaatacagagtgtgttttaccaaaacgcaaaccatgtcacgtagaagacagcggctatggcaatagtgaagtggaagcgcagcagatggcagaccaggtagacgggcaaaatggcgactggcaaagtaacagtagtcaatcaagtggggtgggggctagtaattcagatgtaagttgtactagtatagaggacaatgaggaaaatagtaatagaacgctaaaaagcatacaaaatattatgtgcgaaaatagcataaaaacaactgtattatttaaatttaaagaaacatatggtgttagctttatggaattagtaagaccatttaaaagtaatagaagtagttgtacagattggtgtattataggaatgggagtaacaccatcagttgcagaaggattaaaagtattaatacagccctatagcatatatgcccatttgcaatgtttaacatgtgacagaggcgtgcttatactgctgctaattaggtttaaatgtggaaaaaacagattaacagtgtccaaactaatgtcacagctgttaaatataccagaaacacatatggtaatagaaccaccaaaattacgaagtgctacctgtgcattatattggtatagaacaggtttgtctaatattagtgaggtatatggtaccaccccagaatggatagaacaacaaacagtattacagcatagctttgacaatagcatattcgattttggagaaatggtgcaatgggcatatgatcatgatataacagatgatagtgacatagcatataaatatgcacagttagcagatgtaaatagcaatgctgcagcattcctaaaaagcaattcgcaagcaaaaatagtaaaggactgtgcaaccatgtgtagacattataaacgggcagaaagaaaacatatgaatattggacaatggatacagtatagatgtgatagaatagatgatggtggagattggaggcctatagtaagatttttaagatatcaagacatagaatttacagcctttttagacgcatttaaaaaatttttaaaaggtatacctaaaaaaaattgtttagtattatatggacctgcaaacacaggaaaatcatattttggaatgagtttaattaggttcttaagtggatgtgtaatatcctatgtaaactcaaaaagccatttttggctacaaccattaacagatgcaaaagtgggtatgatagatgatgtaacacctatatgttggacatatatagatgattatatgagaaatgcactggatggaaatgatatatcagtagatgtaaagcatagagccttagtacaaataaaatgcccaccattaattttaacaacaaatacaaatgcaggaacagatcctaggtggccatatttacatagtagattggttgtgtttcatttcaaaaacccatttccatttgatgaaaatggcaatcctatatatgaaattaacaacgaaaattggaaatcctttttctcaaggacgtggtgcaaattagatttaatacaggaagaggacaaggaaaacgatggagtcgataccggcacgtttaaatgcagtgcaggaaaaaatactagatctatacgaagctgatagtaatgacctaaacgcacaaattgaacattggaaattgactcgaatggaatgtgttttgttttacaaagcaaaggaactgggaataactcatataggccaccaggtggtgccaccaatggcagtgtctaaggcaaaggcctgccaagctattgaactacaattggcattggaggcattaaacaaaacacaatatagcacagatggatggacattacaacaaacaagtctagaaatgtggcgtgcagaaccacaaaaatactttaaaaaacatgggtatacaataacagtgcaatacgataatgataaaaacaatactatggattatacaaactggaaggaaatttatttacttggtgagtgtgaatgtacaattgtagaaggacaagtagattactatgggttatattattggtgtgatggagaaaaaatatattttgtaaaatttagtaacgatgcaaagcaatattgtgtaacaggagtatgggaagtacatgtgggtggtcaggtaattgtttgtcctgcatctgtatctagtaacgaagtatccactactgaaactgctgtccacctatgcaccgaaacctccaagacctccgcagtgtccgtgggtgccaaagacacacacctacaaccaccacagaaacgacgacgaccagacgtcacagactccagaaacaccaagtaccccaacaaccttttgcggggacaacaatccgtggacagtactacacggggactcgtcactgcaactgagtgcacaaacaaaggacgggttgcacatacaacttgtactgcacctataatacacctaaaaggtgatcctaatagtttaaaatgtttaagatatagggtaaaaacacataaaagtttgtatgttcaaatttcatctacctggcattggaccagtaatgaatgtacaaataataaactaggtattgtaacaataacgtacagtgatgaaacacaacgtcaacaatttttaaaaactgttaaaataccaaatactgtgcaagttatacaaggtgtcatgtcattgtgatatttgtacatatgtatatatgtatatgtgtatggtaaacacccaacacaagccaatattgctgctattgtgtatatataacaatgttaggattatttgtattttgttttattttgcttatggtgttttgtgcagtgcttaggccgctcttgctatctatatcggtgtatgcgcaggtgttggtgctggtgcttttgctatgggtatctattgggtcaccatttaaagtgttttttttgtacctactgtttttatattttccaatgttttgtattcactgtcatgcacagtatttggcacaactgcaataactgtacatgtagattggctacatgcatatatgcaaaatatactttttcacttttgtagtttgtctaataaatacttttatattttttaatagcttgtcgcaatgagatacagacggtctacacggcacaaacgtgcttctgcaacacagctatatcaaacatgcaaagcctctggcacctgcccccccgatgttattcctaaagtggaaggcacaactattgcagatcaacttttaaaatatggcagcctaggggtgttttttggaggtttgggtataggtacaggtgcaggctctggtggtagggcaggctatgtgccattgtccactcgtcctcccactagtagtattaccacgtccaccattcgtccccctgtaactgtagaacccattggtcccttagaaccatctatagtttctatgatagaagaaacaacatttattgagtctggcgcacctgctccatctattccatcagcaacagggtttgatgttacaacatctgcaaataatactcctgcaataattaatgtaacatctataggtgaatcatctgtacaatcagtttctacacatttaaatcctacattcactgaaccatctataatacagcccccggcacctgcagaagcatctggtcatgtattgttttctagtccaactattagtacacacacctatgaagaaatccctatggatacatttgttacctctactgacagcagcagtgtaacaagtagtacacctattccagggtctcgccctacgacacgccttggtttatatagccgtgccacacaacaggttaaggtagtcgaccctgcttttatgtcatcaccacagaaattagtaacatataacaatcctgtttttgagggcgttgatacagatgaaactataatttttgatcgttcacaacttttacctgcaccggatcctgattttttagacattatagctttgcataggcctgcattaacctctcgaagaggtactgttaggtttagcaggcttggtaataaggccaccctacgtacacgtagtggaaaacaaattggggcacgggtacattattatcatgatattagtcctatccagcctgctgaagttcaggaagacatagaattgcaacctttattaccacagtctgtgtccccttacactattaatgatggtttgtatgatgtgtatgcagattctttgcagcaacccacgtttcacttaccttccacactttctacccataataatactttcactgtacctattaatagtggtattgactttgtatatcaacccactatgtccattgagtcaggtcctgacattccattaccttcgttacccacacatactccttttgttcctatagcccctacagctccatctacatctattattgttgatggtacagattttattttacatcctagttattttttactacgtcgcaggcgtaaacgttttccatatttttttacagatgtccgtgtggcggcctagtgaggccactgtgtacctgcctcctgtacctgtctctaaggttgtaagcactgatgagtatgtgtctcgcacaagcatctattattatgcaggcagttctcgattactaacagtaggacatccctatttttctattaaaaacaccagtagtggtaatggtaaaaaagttttagttcccaaggtgtctggcctgcaatacagggtatttagaattaaattgccggaccctaataaatttggttttccagatacatctttttataacccagaaacccaaaggttggtgtgggcctgtacaggcttggaaattggtaggggacagcctttaggtgtgggtattagtgggcatcctttattaaacaagtttgatgatactgaaaccagtaacaaatatgctggtaaacctggtatagataatagggaatgtttatctatggattataagcagactcagttatgcattttaggatgcaaacctcctataggtgaacattggggtaagggaaccccttgtaataataattcaggaaatcctggggattgtcctcccctacagctcattaacagtgtaatacaggatggggacatggtagatacaggatttggttgcatggattttaataccttgcaagctagtaaaagtgatgtgcccattgatatatgtagcagtgtatgtaagtatccagattatttgcaaatggctagcgagccatatggtgacagtttgttcttttttcttagacgtgagcaaatgtttgttagacacttttttaatagggccggtaccttaggtgaccctgtgccaggtgatttatatatacaagggtctaactctggcaatactgccactgtacaaagcagtgctttttttcctactcctagtggttctatggtaacctcagaatcccaattatttaataaaccgtactggttacaacgtgcgcagggccacaataatggcatatgttggggcaatcagttgtttgtcacagttgtggataccactcgtagcactaacatgactttatgtgctgaggttaaaaaggaaagcacatataaaaatgaaaattttaaggaataccttcgtcatggcgaggaatttgatttacaatttatttttcaattgtgcaaaattacattaacagctgatgttatgacatacattcataagatggatgccactattttagaggactggcaatttggccttaccccaccaccgtctgcatctttggaggacacatacagatttgtcacttctactgctataacttgtcaaaaaaacacaccacctaaaggaaaggaagatcctttaaaggactatatgttttgggaggtggatttaaaagaaaagttttctgcagatttagatcagtttcctttaggtaggaagtttttgttacaggcagggctacaggctaggcccaaactaaaacgccctgcatcatcggccccacgtacctccacaaagaagaaaaaggttaaaaggtaaccattgtctgttgggtaattgtctgtgtcatgtatgtgttgtgtatgtcaaacacaggttaaaaggtaaccattgtttgttatgtaattgttttgtgtgtgtactgtgttgtttgcatgttatgtatgtgtgtgcatgtttgttgtatttgtcagttcctgtatgtatgttttgtgtatgtattaataaagtactgtatttactaaactatttatagtagtcttatgttatgttatggttgcacccacatgagtaacaatacagttgctcctaatctattgcatctcctgccctaccctgtgtcccctgccctaccctgtgtcctactttgttacactactaattagccttatactctccattttgtaccattttgtactatccaccattttaaatcctaaccgaattcggttggtcttggcacaactttggttgtccttggcacagtaacaactatttttatataagtttcagcaaactgcttaatcctttggtttcctgcagtccactggtctacacttgttgtcccgcctaaactgacttcttgctgactcacaggtcctgcagtgcagctaaacaatacattgcctaacattgcatgttttaaactgcttttaggcacatattttatttaaactttcaatgcactaattacagtgttggcttacacaagtacatcctacgccaaatatgtcttgtaaaacatgattaaatactgttactcaccaggtgtgcactacacgaccggttacggttaccgtacccacaaccacttttttttataatta

SEQ ID NO: 3 - DNA, 7824 bp, Human papillomavirus type 58
ctaaactataatgccaaatcttgtaaaaactagggtgtaaccgaaaacggtctgaccgaaaccggtgcatatataaagcagacattttttggtaggctactgcaggactatgttccaggacgcagaggagaaaccacggacattgcatgatttgtgtcaggcgttggagacatctgtgcatgaaatcgaattgaaatgcgttgaatgcaaaaagactttgcagcgatctgaggtatatgactttgtatttgcagatttaagaatagtgtatagagatggaaatccatttgcagtatgtaaagtgtgcttacgattgctatctaaaataagtgagtatagacattataattattcgctatatggagacacattagaacaaacactaaaaaagtgtttaaatgaaatattaattagatgtattatttgtcaaagaccattgtgtccacaagaaaaaaaaaggcatgtggatttaaacaaaaggtttcataatatttcgggtcgttggacagggcgctgtgcagtgtgttggagaccccgacgtagacaaacacaagtgtaacctgtaacaacgccatgagaggaaacaacccaacgctaagagaatatattttagatttacatcctgaaccaactgacctattctgctatgagcaattatgtgacagctcagacgaggatgaaataggcttggacgggccagatggacaagcacaaccggccacagctaattactacattgtaacttgttgttacacttgtggcaccacggttcgtttgtgtatcaacagtacaacaaccgacgtacgaaccctacagcagctgcttatgggcacatgtaccattgtgtgccctagctgtgcacagcaataaacaccatctgcaatggatgaccctgaaggtacaaacggggtaggggcgggctgtactggctggtttgaggtagaagcggtaatagaacgaagaacaggagataatatttcagatgatgaggacgaaacagcagacgatagtggtacagatttaatagagtttatagatgattcagtacaaagtactacacaggcagaagcagaggcagcccgagcgttgtttaatgtacaggaaggggtggacgatataaatgctgtgtgtgcactaaaacgaaagtttgcagcatgctcagaaagtgctgtagaggactgtgtggaccgggctgcaaatgtgtgtgtatcgtggaaatataaaaataaagaatgcacacacagaaaacgaaaaattattgagctagaagacagcggatatggcaatactgaagtggaaactgagcagatggcacaccaggtagaaagccaaaatggcgacgcagacttaaatgactcggagtctagtggggtgggggctagttcagatgtaagcagtgaaacggatgtagacagttgtaatactgttccattacaaaatattagtaatattctacataacagtaatactaaagcaacgctattatataaattcaaagaagcttatggagtaagttttatggaattagttagaccatttaaaagtgataaaacaagctgtacagattggtgtataacagggtatggaataagtccctccgtagcagaaagtttaaaagtactaattaaacagcacagtatatatacacacctacaatgtttaacgtgtgacagaggaattatattattattgttaattagatttaaatgtagcaaaaatagattaactgtggcaaaattaatgagtaatttactatcaattcctgaaacatgtatgattatcgagccaccaaaattacgaagtcaagcatgtgccttatattggtttagaacagcaatgtcaaatataagtgatgtgcaagggacaacaccagaatggatagatagattaacagtgttacagcatagctttaatgatgatatatttgatttaagtgaaatgatacaatgggcatatgataatgacattacagatgatagtgacattgcatataaatatgcacagttagcagatgttaatagtaatgcagcagcatttttaagaagcaatgcacaagcaaaaatagtaaaagactgtggcgttatgtgcagacattataaaagagcagaaaagcgtggtatgacaatgggacaatggatacaaagtaggtgtgaaaaaacaaatgatggaggtaattggagaccaatagtacaatttttaagatatcaaaatattgaatttacagcatttttagttgcatttaaacagtttttacaaggtgtaccaaaaaaaagttgtatgttactgtgtggcccagcaaatacagggaaatcatattttggaatgagtttaatacattttttaaaaggatgcattatttcatatgtaaattccaaaagtcatttttggttgcagccattatcagatgctaaactaggtatgatagatgatgtaacagccataagctggacatatatagatgattatatgagaaatgcattagatggtaacgacatttcaatagatgtaaaacatagggcattagtacaattaaaatgtccaccattaataattacctcaaatacaaatgcaggcaaagattcacgatggccatatttgcacagtagactaacagtatttgaatttaacaatccatttccatttgatgcaaatggtaatccagtgtataaaataaatgatgaaaattggaaatcctttttctcaaggacgtggtgcaaattaggcttaatagaggaagaggacaaggaaaacgatggaggaaatatcagcacgtttaagtgcagtgcaggacaaaatcctagacatatacgaagctgataaaaatgatttaacatcacaaattgaacattggaaactaatacgcatggagtgtgctataatgtatacagccagacaaatgggaatatcacatttgtgccaccaggtggtgccgtcattggtagcatcaaagactaaagcgtttcaagtaattgaactgcaaatggcattagagacattaaatgcatcaccatataaaacagatgaatggacattgcaacaaacaagcttagaagtgtggttatcagagccacaaaaatgctttaaaaaaaaaggcataacagtaactgtacaatatgacaatgataaagcaaacacaatggattatacaaattggagtgaaatatatattattgaggaaacaacatgtactttggtagcaggagaagttgactatgtggggttgtattatatacatggcaatgaaaagacgtattttaaatattttaaagaggatgcaaaaaagtactctaaaacacaattatgggaggtacatgtgggtagtcgggtaattgtatgtcctacatctatacctagtgatcaaatatccactactgaaactgctgacccaaagaccaccgaggccaccaacaacgaaagtacacaggggacaaagcgacgacgactcgatttaccagactccagagacaacacccagtactccacaaagtatacagactgcgccgtggacagtagaccacgaggaggaggactacacagtacaactaactgtacatacaaagggcggaacgtgtgtagttctaaagtttcacctatcgtgcatttaaaaggtgacccaaatagtttaaaatgtttaagatatagattaaaaccatttaaagacttatactgtaatatgtcatccacatggcattggaccagtgatgacaaaggtgacaaagtaggaattgttactgtaacatacacaacggaaacacaacgacaactgtttttaaacactgttaaaataccacccactgtgcaaataagtactggtgttatgtcattgtaattgtattgtacaattactgtatgtaaaccacaagccaatatgtgctgctaagtgtatatacaatgatattacctatttttgttgtttgttttatactgtttttatgcttgtgcatttttttgcggccattggtgctatctatttctatatatgcttggttgctggtgttggtgttgctgctttgggtgtctgtggggtcggctctacgaatttttttctgttacttaatatttttatatataccaatgatgtgtattaattttcatgcacaatacttaacccaacaagactaactgtatactggttctgcacatggtggtatggtattgtaaatatttactgttgtgtgtgttgtttttattatttttatacatttactaataaatacttttatatttttagcactgtcttattatgagacacaaacggtctacaaggcgcaagcgtgcatctgctacacaactttaccaaacatgcaaggcctcaggcacctgcccacctgatgttatacccaaagttgaaggcactactatagcagatcaaatattacgatatggtagcttaggggtgttttttggaggtttaggcattggtacagggtcgggtacaggtggcaggactggatatgtgccccttggtagtaccccaccgtctgaggctatacctttacagcccatacgtcccccagttaccgttgatactgtggggcctttggattcttctattgtatctttaatagaggaatctagttttatagacgccggtgcaccagccccatcaattcccactccatctggttttgatattaccacctctgcagatactacacctgcaatacttaatgtttcctctattggagaatcatctatacaaactgtttctacacatttaaatccctcctttactgagccatccgtactccgccctcctgcacctgcagaggcctctggacatttaatattttcctctcctactgttagcacacatagttatgaaaacataccaatggatacctttgttatttctactgacagtggcaatgtcacgtctagcacacccattccagggtctcgccctgtggcacgccttggtttatacagtcgcaacacccaacaagttaaggttgttgaccctgcttttttaacatctcctcatagacttgtaacatatgataatccagcatttgaaggctttaaccctgaggacacattgcagtttcaacatagtgacatatcgcctgctcctgatcctgattttctagatattgttgcattacacagacctgcattaacctctcgcaggggtactgtacgttatagtagggttgggcaaaaggctacacttcgtactcgcagtggaaagcaaataggggctaaagtacattactaccaagacttaagtcccatacagcctgtccaggaacaggtacaacagcagcaacaatttgaattacaatctttaaatacttctgtttctccctatagtattaatgatggactttatgatatttatgctgacgatgctgatactatacatgattttcagagtcctctgcactcacatacgtcctttgccaccacacgtaccagtaatgtgtccataccattaaatactggatttgacactcctcttgtgtcattggaacctggtccagacattgcatcttctgtaacatctatgtctagtccatttattcctatatctccactaactccttttaataccataattgtggatggtgctgattttatgttgcaccctagctattttattttgcgtcgcagacgtaaacgttttccatatttttttgcagatgtccgtgtggcggcctagtgaggccactgtgtacctgcctcctgtgcctgtgtctaaggttgtaagcactgatgaatatgtgtcacgcacaagcatttattattatgctggcagttccagacttttggctgttggcaatccatatttttccatcaaaagtcccaataacaataaaaaagtattagttcccaaggtatcaggcttacagtatagggtctttagggtgcgtttacctgatcccaataaatttggttttcctgatacatctttttataaccctgatacacaacgtttggtctgggcatgtgtaggccttgaaataggtaggggacagccattgggtgttggcgtaagtggtcatccttatttaaataaatttgatgacactgaaaccagtaacagatatcccgcacagccagggtctgataacagggaatgcttatctatggattataaacaaacacaattatgtttaattggctgtaaacctcccactggtgagcattggggtaaaggtgttgcctgtaacaataatgcagctgctactgattgtcctccattggaactttttaattctattattgaggatggtgacatggtagatacagggtttggatgcatggactttggtacattgcaggctaataaaagtgatgtgcctattgatatttgtaacagtacatgcaaatatccagattatttaaaaatggccagtgaaccttatggggatagtttgttcttttttcttagacgtgagcagatgtttgttagacacttttttaatagggctggaaaacttggcgaggctgtcccggatgacctttatattaaagggtccggtaatactgcagttatccaaagtagtgcattttttccaactcctagtggctctatagttacctcagaatcacaattatttaataagccttattggctacagcgtgcacaaggtcataacaatggcatttgctggggcaatcagttatttgttaccgtggttgataccactcgtagcactaatatgacattatgcactgaagtaactaaggaaggtacatataaaaatgataattttaaggaatatgtacgtcatgttgaagaatatgacttacagtttgtttttcagctttgcaaaattacactaactgcagagataatgacatatatacatactatggattccaatattttggaggactggcaatttggtttaacacctcctccgtctgccagtttacaggacacatatagatttgttacctcccaggctattacttgccaaaaaacagcaccccctaaagaaaaggaagatccattaaataaatatactttttgggaggttaacttaaaggaaaagttttctgcagatctagatcagtttcctttgggacgaaagtttttattacaatcaggccttaaagcaaagcccagactaaaacgttcggcccctactacccgtgcaccatccaccaaacgcaaaaaggttaaaaaataattgttgtggtacttacactattttattatacatgtttgtttgttttatgtatgtgttgtctgtttgtttatgtttgtgtatatgttgtatgtgttatgtgtcatgtttgtgtacatgttctatgtccttgtcagtttcctgtttctgtatatatgtaataaactattgtgtgtattgtaaactatttgtattgtttgggtgtatctatgagtaaggtgctgtccctaaattgccctaccctgccctgcctattatgcatacctatgtaatagtatttgtatgatatgtattttatagtttttaacagtactgcctccattttactttacctccattttgtgcatgtaaccgatttcggttgctggcacaaacgtgttttttttaaactacaatttaaacaatacagttaatcctttcccttcctgcactgcttttgcctatacttgcatatgtgactcatatatacatgcagtgcagttgcaaaatgtttaattatactcatagtttaaacatgcttataggcacatattttaacttactttcaatgcttaagtgcagttttggcttgcacaatagtttgttatgccaaactatgtcttgtaaaagtgactcactaacatttattgccaggtgtggactaaccgttttgggtcacattgttcatgtttcaacattttatataata

SEQ ID NO: 4 - DNA, 35 bp, Artificial
  PCR Primer
ttaatatttattaattgtgttgtggttatttattg

SEQ ID NO: 5 - DNA, 35 bp, Artificial
  PCR Primer
taaccttaaaaatttaaaccttataccaaatatac

SEQ ID NO: 6 - DNA, 30 bp, Artificial
  PCR Primer
agtaggattgaaggttaaattaaaatttat

SEQ ID NO: 7 - DNA, 30 bp, Artificial
  PCR Primer
aacacattttataccaaaaaacatacaacc

SEQ ID NO: 8 - DNA, 27 bp, Artificial
  PCR Primer
aatagggttggtattgttggtgaaaat

SEQ ID NO: 9 - DNA, 30 bp, Artificial
  PCR Primer
ttccaatcctccaaaataataaaattcata

SEQ ID NO: 10 - DNA, 31 bp, Artificial
  PCR Primer
gtattattttgtattatttattattttaaat

SEQ ID NO: 11 - DNA, 33 bp, Artificial
  PCR Primer
ccctattttttactaatataaaattataattta

SEQ ID NO: 12 - DNA, 30 bp, Artificial
  PCR Primer
tttatttatatttattattgttgatggtat

SEQ ID NO: 13 - DNA, 25 bp, Artificial
  PCR Primer
taaaaaaccaaatttattaaaatcc

SEQ ID NO: 14 - DNA, 23 bp, Artificial
  PCR Primer
attttagaggattggtaatttgg

SEQ ID NO: 15 - DNA, 26 bp, Artificial
  PCR Primer
aacctttttcttctttataaaaatac

SEQ ID NO: 16 - DNA, 26 bp, Artificial
  PCR Primer
ttttatttttattttgtgtatgtaat

SEQ ID NO: 17 - DNA, 25 bp, Artificial
  PCR Primer
taatcctacaataacctaccaaaaa

SEQ ID NO: 18 - DNA, 24 bp, Artificial
  PCR Primer
atggtgttgattttatgttgtatt

SEQ ID NO: 19 - DNA, 25 bp, Artificial
  PCR Primer
aactatcccctacctatttcaaaac

SEQ ID NO: 20 - DNA, 24 bp, Artificial
  PCR Primer
ttaatattttggaggattggtaat

SEQ ID NO: 21 - DNA, 30 bp, Artificial
  PCR Primer
atataataaaataatataaataccacaaca

SEQ ID NO: 22 - DNA, 29 bp, Artificial
  PCR Primer
aattaggttttaaagtaaagtttagatta

SEQ ID NO: 23 - DNA, 29 bp, Artificial
  PCR Primer
ttatttaaattataatttaaaaaaaacac

SEQ ID NO: 24 - DNA, 36 bp, Artificial Sequence
  PCR Primer
ttgttgatgtaggtgatttttatttatattttagtt

SEQ ID NO: 25 - DNA, 29 bp, Artificial Sequence
  PCR Primer
ccactaatacccacacctaataactaacc

SEQ ID NO: 26 - DNA, 22 bp, Artificial Sequence
  PCR Primer
tttaggtgattttgtgttaggt

SEQ ID NO: 27 - DNA, 25 bp, Artificial Sequence
  PCR Primer
tcctctaaaataataacatccatct

SEQ ID NO: 28 - DNA, 24 bp, Artificial Sequence
  PCR Primer
ttggttgtatggattttaatattt

SEQ ID NO: 29 - DNA, 21 bp, Artificial Sequence
  PCR Primer
aacaaacaactaattacccca

SEQ ID NO: 30 - DNA, 21 bp, Artificial Sequence
  PCR Primer
tggttagtgaattttatgggg

SEQ ID NO: 31 - DNA, 30 bp, Artificial Sequence
  PCR Primer
ttacaaaactaaaaaacaaactataaatca

SEQ ID NO: 32 - DNA, 31 bp, Artificial Sequence
  PCR Primer
gatgtaggtgatttttatttatattttagtt

SEQ ID NO: 33 - DNA, 27 bp, Artificial Sequence
  PCR Primer
atccaactacaaataatctaaatattc

SEQ ID NO: 34 - DNA, 30 bp, Artificial Sequence
  PCR Primer
gattatgtattttgggaggttaatttaaaa

SEQ ID NO: 35 - DNA, 30 bp, Artificial Sequence
  PCR Primer
cataacatacatacacacataacatactat

SEQ ID NO: 36 - DNA, 28 bp, Artificial Sequence
  PCR Primer
ggtgattgttttttattagaattaaaaa

SEQ ID NO: 37 - DNA, 26 bp, Artificial Sequence
  PCR Primer
aataccattattatatccctaaacac

SEQ ID NO: 38 - DNA, 33 bp, Artificial Sequence
  PCR Primer
gttatttgatttaaataaatttggtttatttga

SEQ ID NO: 39 - DNA, 27 bp, Artificial Sequence
  PCR Primer
tccataacaccatatccaatatctacc

SEQ ID NO: 40 - DNA, 25 bp, Artificial Sequence
  PCR Primer
tggtagttttagattttttgttgtt

SEQ ID NO: 41 - DNA, 21 bp, Artificial Sequence
  PCR Primer
ctttaccccaatattccccta

SEQ ID NO: 42 - DNA, 25 bp, Artificial Sequence
  PCR Primer
tttttagtggttttatggtaatttt

SEQ ID NO: 43 - DNA, 29 bp, Artificial Sequence
  PCR Primer
aaatttttaacaaattacaacctataata

(19) United States
(12) Patent Application Publication
(10) Pub. No.: US 2011/0189656 A1
(43) Pub. Date: Aug. 4, 2011

(54) Method for Determining Presence or Absence of Abnormal Cell

(76) Inventors: Noriko Oka, Hyogo (JP); Masahiro Kajita, Hyogo (JP)

(21) Appl. No.: 13/124,771

(22) PCT Filed: Sep. 29, 2009
(86) PCT No.: PCT/JP2009/066963

§ 371 Date: Apr. 18, 2011

(30) Foreign Application Priority Data
Oct. 23, 2008 (JP) 2008-273257

(51) Int. Cl. C12Q 1/70; C07H 21/00

(52) U.S. Cl. 435/5; 536/24.33

(57) ABSTRACT

A method for determining the presence or absence of an abnormal cell in a sample collected from the uterine cervix of a subject, and a method for predicting the progression of a lesion in the uterine cervix in a subject, each of which comprises measuring the frequency of methylation in the genomic DNA of human papillomavirus contained in the sample and determining the presence or absence of the abnormal cell or predicting the progression of the lesion based on the frequency; and a primer set which can be used in the above-mentioned methods.

### TECHNICAL FIELD

The present invention relates to methods for determining whether or not abnormal cells originated from severe dysplasia or lesion in more advanced stages of uterine cervix are contained in a sample obtained from uterine cervix of a subject and for predicting whether or not uterine cervical tissue progresses to severe dysplasia or lesion in more advanced stages, by detecting methylation of genomic DNA of human papillomavirus (hereinafter also referred to as “HPV”) in the sample, and to a primer set used for the methods.

### BACKGROUND ART

It has been known that the infection with HPV accounts for a significant part of risk factors for cervical cancer. In most cases, the infection with HPV is transient in which HPV spontaneously disappears from the infected cells in a certain period after the infection. However, HPV does not disappear from 5 to 10% of the patients infected with HPV, producing a persistent infection and resulting in the development of cervical cancer.

Cervical cancer is developed in the uterine cervical surface epithelium, and is mostly squamous cell cancer. In the conventional examinations for cervical cancer, cells taken from uterine cervix are subjected to the screening by cytological diagnosis. When the cells are diagnosed to be abnormal, then detailed examinations such as histological diagnosis are carried out.

In histological diagnosis of uterine cervix, the cells are classified into three stages, i.e. mild, moderate and severe dysplasias, as the premalignant stages, according to the extent of the emergence of atypical cells in epithelium. When lesion is exacerbated beyond severe dysplasia, it comes to the stage in which cancer cells appear in epithelium. The lesion proceeds to “intraepithelial cancer” in which cancer cells are localized in epithelium and then to “microinvasive squamous cancer” and “invasive squamous cancer” in which cancer cells invade from epithelium to subcutaneous tissue.

Most of mild and moderate dysplasias disappear. In order to avoid overtreatment, most of such lesions are followed-up without any treatment. However, as antecedent lesions of severe dysplasia are highly possible to progress to invasive cancer without any treatment, the patients diagnosed as severe dysplasia may frequently undergo treatment such as surgical operations.

The results of pathological diagnosis by histological diagnosis may vary depending on skill of examiners. Thus, the reproducibility of pathological diagnosis is low; due to this, in some cases, patients who are diagnosed as moderate dysplasia in pathological diagnosis may receive treatment such as surgical operations in fear of delay in the start of treatment for severe dysplasia and invasive cancer. In such case, although delay in the start of treatment may be prevented, there is a possibility for overtreatment.

Therefore, it is important to determine whether or not lesion of a subject is severe dysplasia or in more advanced stages, in order to determine treatment strategies for the subject.

Even when a patient is diagnosed as mild or moderate dysplasia by histological diagnosis of uterine cervix, he/she may later progress to severe dysplasia in some cases. Of course, it is important in view of early treatment to predict beforehand whether lesion will progress to severe dysplasia. However, it was difficult to make such prediction with conventional diagnosis methods.

Histological diagnosis of uterine cervix is carried out by collecting a small amount of uterine cervical tissue with biopsy from a subject who has been determined to be in need by a screening cytological diagnosis, and microscopically observing the tissue stained with hematoxylin-eosin (HE) staining.

Histological diagnosis requires the decision by examiners based on the observations, and cervical cancer may be overlooked depending on for instance the way of preparation of samples to be examined. In addition, because it requires a great deal of skill of examiners, it is difficult to promptly and accurately examine large number of samples.

HPV has a circular double-stranded DNA as a genome and is classified into more than 100 subtypes. Among these subtypes, those highly possible to cause cervical cancer are classified as high-risk HPV. Specific high risk-HPV includes HPV-16, HPV-18, HPV-31, HPV-33, HPV-35, HPV-39, HPV-45, HPV-51, HPV-52, HPV-56, HPV-58, HPV-59, HPV-68, HPV-73 and HPV-82. Among these, HPV-16, HPV-18, HPV-52 and HPV-58 are known as high-risk species which are liable to be detected from patients who have developed cervical cancer. Genomic DNA of HPV contains the regions conserved among subtypes such as L1 region, L2 region and LCR which encode capsid proteins and E1, E2, E4, E5, E6 and E7 regions which encode non-structural proteins.

Recently, T. Turan et al. reported that methylation of 5′-(CG)-3′ (CpG) in genomic DNA of HPV such as in L1 region, LCR may be used as an index for detecting cervical cancer (Non Patent Literature 1). T, Turan et al. disclose that methylation in L1 region is detected only in cancer samples with the exception of some antecedent lesions, so that the detection of methylation in L1 region may be an important molecular marker for cancer diagnosis (see Abstract, for example).

WO 2008/071998 (Patent Literature 1) discloses that overmethylation in HPV genome in the samples from patients indicates the progression of diseases caused by HPV infection. Patent Literature 1 specifically discloses the detection of methylation in L2 and E2 regions of HPV genomic DNA.

Patent Literature 1: WO 2008/071998

Non Patent Literature 1: T. Turan et al., “Methylation of human papillomavirus-18 L1 gene: A biomarker of neoplastic progression?” Virology 349 (2006) p. 175-183

### SUMMARY OF THE INVENTION

### Technical Problem

The present invention aims to provide a method which allows more accurate and easier determination on whether or not uterine cervical lesion of a subject is severe dysplasia or lesion in more advanced stages.

The present invention also aims to provide a method which allows more accurate and easier prediction on whether or not uterine cervical lesion of a subject will progress to severe dysplasia or lesion in more advanced stages.

The present invention further aims to provide a primer set which is used for the above methods of detection and determination.

### Solution to Problem

The present inventors have measured the frequency of methylation in L1 region of HPV genomic DNA in the samples obtained from uterine cervix of the patients who have been diagnosed as having lesions in various stages by histological diagnosis. As a result, they have found that the frequency of methylation in L1 region can be an index for determination on whether the lesion is severe dysplasia or lesion in more advanced stages, or is in less advanced stages than severe dysplasia. Further, they have found that the frequency of methylation in L1 region may be an index for determination on whether the lesion which has been diagnosed as in less advanced stages than severe dysplasia will progress to severe dysplasia or lesion in more advanced stages, and completed the present invention.

Thus, the present invention provides:

(1) a method of determining the presence or absence of abnormal cells originated from severe dysplasia or lesion in more advanced stages of uterine cervix in a sample obtained from uterine cervix of a subject, comprising the steps of: measuring a frequency of methylation of 5′-(CG)-3′ (CpG) in L1 region of HPV genomic DNA in the sample; and determining whether or not the abnormal cells are contained in the sample based on the measured frequency of methylation;

(2) the method according to (1), wherein, in the step of determining, the measured frequency of methylation is compared to a predetermined threshold and the sample is determined to contain the abnormal cells when the frequency of methylation is higher than the threshold;

(3) a method of predicting whether or not uterine cervix tissue of a subject progresses to severe dysplasia or lesion in more advanced stages, comprising the steps of: measuring a frequency of methylation of 5′-(CG)-3′ (CpG) in L1 region of HPV genomic DNA in a sample obtained from uterine cervix of the subject; and predicting whether or not the tissue progresses to severe dysplasia or lesion in more advanced stages based on the measured frequency of methylation;

(4) the method according to (3), wherein, in the step of predicting, the measured frequency of methylation is compared to a predetermined threshold and the tissue is predicted to progress to severe dysplasia or lesion in more advanced stages when the frequency of methylation is higher than the threshold;

(5) the method according to any one of (1) to (4), wherein the frequency of methylation is obtained by dividing the number of methylated CpG(s) present in said L1 region which is subjected to the measurement of the frequency of methylation by the number of all CpGs present in said L1 region;

(6) the method according to any one of (1) to (5), wherein said L1 region which is subjected to the measurement of the frequency of methylation is a region which comprises at least one CpG existing within 80% from 5′-terminal among all CpGs in the L1 region;

(7) the method according to any one of (1) to (6), wherein HPV is at least one selected from HPV-16, HPV-18, HPV31, HPV33, HPV35, HPV-52 and HPV-58;

(8) the method according to (7), wherein HPV is HPV-16 and wherein said L1 region which is subjected to the measurement of the frequency of methylation is a region which comprises at least one CpG among the 1^{st} to 15^{th} CpGs from 5′-terminal of L1 region of HPV-16 and does not comprise the 16^{th} to 19^{th} CpGs;

(9) the method according to (7), wherein HPV is HPV-31 and wherein said L1 region which is subjected to the measurement of the frequency of methylation is a region which comprises at least one CpG among the 1^{st} to 17^{th} CpGs from 5′-terminal of L1 region of HPV-31 and does not comprise the 18^{th} to 22^{nd} CpGs;

(10) the method according to (7), wherein HPV is HPV-52 and wherein said L1 region which is subjected to the measurement of the frequency of methylation is a region which comprises at least one CpG among the 1^{st} to 17^{th} CpGs from 5′-terminal of L1 region of HPV-52 and does not comprise the 18^{th} to 22^{nd} CpGs;

(11) the method according to (7), wherein HPV is HPV-58 and wherein said L1 region which is subjected to the measurement of the frequency of methylation is a region which comprises at least one CpG among the 1^{st} to 19^{th} CpGs from 5′-terminal of L1 region of HPV-58 and does not comprise the 20^{th} to 25^{th} CpGs;

(12) the method according to any one of (1) to (11), wherein severe dysplasia or lesion in more advanced stages includes severe dysplasia, intraepithelial cancer, microinvasive squamous cancer and invasive squamous cancer;

(13) a primer set for determining the presence or absence of abnormal cells originated from severe dysplasia or lesion in more advanced stages of uterine cervix, or for predicting the progress to severe dysplasia or lesion in more advanced stages, which is used in a nucleic acid amplification method for amplification of a region comprising at least one CpG existing within 80% from 5′-terminal among all CpGs in L1 region of HPV genomic DNA, said region having been treated with bisulfite;

(14) the primer set according to (13), wherein HPV is at least one selected from HPV-16, HPV-18, HPV-31, HPV33, HPV35, HPV-52 and HPV-58;

(15) the primer set according to (14), wherein HPV is HPV-16 and wherein the region amplified in the amplification method is a region which comprises at least one CpG among the 1^{st} to 15^{th} CpGs from 5′-terminal of L1 region of HPV-16 and does not comprise the 16^{th} to 19^{th} CpGs, said region having been treated with bisulfite;

(16) the primer set according to (14), wherein HPV is HPV-18 and wherein the region amplified in the amplification method is a region which comprises at least one CpG among the 1^{st} to 25^{th} CpGs from 5′-terminal of L1 region of HPV-18 and does not comprise the 26^{th} to 32^{nd} CpGs, said region having been treated with bisulfite;

(17) the primer set according to (14), wherein HPV is HPV-31 and wherein the region amplified in the amplification method is a region which comprises at least one CpG among the 1^{st} to 17^{th} CpGs from 5′-terminal of L1 region of HPV-31 and does not comprise the 18^{th} to 22^{nd} CpGs, said region having been treated with bisulfite;

(18) the primer set according to (14), wherein HPV is HPV-33 and wherein the region amplified in the amplification method is a region which comprises at least one CpG among the 1^{st} to 16^{th} CpGs from 5′-terminal of L1 region of HPV-33 and does not comprise the 17^{th} to 21^{st} CpGs, said region having been treated with bisulfite;

(19) the primer set according to (14), wherein HPV is HPV-35 and wherein the region amplified in the amplification method is a region which comprises at least one CpG among the 1^{st} to 13^{th} CpGs from 5′-terminal of L1 region of HPV-35 and does not comprise the 14^{th} to 17^{th} CpGs, said region having been treated with bisulfite;

(20) the primer set according to (14), wherein HPV is HPV-52 and wherein the region amplified in the amplification method is a region which comprises at least one CpG among the 1^{st} to 17^{th} CpGs from 5′-terminal of L1 region of HPV-52 and does not comprise the 18^{th} to 22^{nd} CpGs; and

(21) the primer set according to (14), wherein HPV is HPV-58 and wherein the region amplified in the amplification method is a region which comprises at least one CpG among the 1^{st} to 19^{th} CpGs from 5′-terminal of L1 region of HPV-58 and does not comprise the 20^{th} to 25^{th} CpGs.

### Advantageous Effect of Invention

According to the present methods in which methylation is simply measured in a specific region of HPV genomic DNA in samples obtained from uterine cervix of subjects, it is possible to carry out the determination on whether or not the samples contain abnormal cells originated from clinically important severe dysplasia or lesion in more advanced stages, or the prediction on whether or not uterine cervical tissue progresses to severe dysplasia or lesion in more advanced stages.

The present methods allow accurate determination or prediction independent of skills of examiners, because the frequency of methylation of HPV genomic DNA in samples is measured.

### BRIEF DESCRIPTION OF THE DRAWINGS

FIGS. 1-1 to 1-10 show the results of detection of methylated CpGs in L1 region or LCR of HPV-16 genomic DNA in samples obtained from subjects.

FIGS. 2-1 to 2-4 show the results of detection of methylated CpGs in L1 region or LCR of HPV-52 genomic DNA in samples obtained from subjects.

FIGS. 3-1 to 3-8 show the results of detection of methylated CpGs in L1 region or LCR of HPV-58 genomic DNA in samples obtained from subjects.

FIGS. 4-1 to 4-4 show total genomic sequence of HPV-16 and the positions of CpGs comprised in L1 region and LCR therein.

FIGS. 5-1 to 5-4 show total genomic sequence of HPV-52 and the positions of CpGs comprised in L1 region and LCR therein.

FIGS. 6-1 to 6-4 show total genomic sequence of HPV-58 and the positions of CpGs comprised in L1 region and LCR therein.

FIG. 7 illustrates the way of calculation of the frequency of methylation.

FIG. 8 is a scatter diagram showing the frequency of methylation in L1 regions of HPV-16, HPV-58 and HPV-52 in relation to the severity of lesion diagnosed by histological diagnosis for operation samples.

FIG. 9 is a scatter diagram of the frequency of methylation of CpGs existing within 80% from 5′-terminal among CpGs in L1 region in relation to the severity of lesion diagnosed by histological diagnosis.

FIG. 10 is a scatter diagram of the frequency of methylation of CpGs existing at 3′-terminal side beyond 80% from 5′-terminal among CpGs in L1 region in relation to the severity of lesion diagnosed by histological diagnosis.

FIG. 11 shows results of histological diagnosis and of determination based on the frequency of methylation of biopsy samples and of histological diagnosis of operation samples obtained from the same subjects.

FIG. 12 is a scatter diagram showing the frequency of methylation (%) in L1 region at the first visit of patients with different courses.

FIG. 13 is a scatter diagram showing the frequency of methylation (%) in L1 region at the first visit of patients in relation to the severity of lesion diagnosed by histological diagnosis.

### DETAILED DESCRIPTION OF THE PREFERRED EMBODIMENTS

As used herein, “frequency of methylation” may indicate a ratio of the number of methylated CpG(s) in an analytical region for methylation. For example, because the number of total CpGs in an analytical region is fixed, the frequency of methylation may be the number of methylated CpG(s) per se in the region. The frequency of methylation can also be a value obtained by dividing the number of methylated CpG(s) in an analytical region by the number of all CpGs in the region. In the present methods, it is preferable that the frequency of methylation is a value obtained by dividing the number of methylated CpG(s) in an analytical region by the number of all CpGs in the region. In such case, the frequency of methylation can be calculated with the following formula I:

(Frequency of methylation) (%)=(the number of methylated CpG(s) in an analytical region)/(the number of all CpGs in an analytical region)×100

The “analytical region” is the whole or partial L1 region of HPV genomic DNA. Preferably, the analytical region is a region which comprises at least one CpG existing within 80% from 5′-terminal among all CpGs in L1 region. The region more preferably does not comprise CpG(s) existing within 20% from 3′-terminal.

By measuring the frequency of methylation of CpGs within such region, it is possible to more accurately carry out the determination on whether or not a sample obtained from uterine cervix contains abnormal cells originated from severe dysplasia or lesion in more advanced stages or the prediction on the progress to severe dysplasia or lesion in more advanced stages of uterine cervix.

More specifically, for L1 region of HPV-16, the analytical region is preferably a region which comprises at least one CpG among the 1^{st} to 15^{th} CpGs from 5′-terminal and does not comprise the 16^{th} to 19^{th} CpGs, among 19 CpGs in total. For L1 region of HPV-52, the analytical region is preferably a region which comprises at least one CpG among the 1^{st} to 17^{th} CpGs from 5′-terminal and does not comprise the 18^{th} to 22^{nd} CpGs, among 22 CpGs in total. For L1 region of HPV-58, the analytical region is preferably a region which comprises at least one CpG among the 1^{st} to 19^{th} CpGs from 5′-terminal and does not comprise the 20^{th} to 25^{th} CpGs, among 25 CpGs in total.

As used herein, “methylated CpG(s)” and “methylation of CpG(s)” mean that cytosine in a consecutive 5′-(CG)-3′ in HPV genomic DNA is methylated at a 5- or 6-position of cytosine base.

As used herein, “L1 region of HPV genomic DNA” is a region defined as L1 region in the sequences which are accessible through public databases showing sequence information of HPV genomic DNA (e.g. GenBank from National Center for Biotechnology Information (NCBI)). For example, total genomic DNA sequence of HPV-16 (GenBank accession number: NC_{—}001526; SEQ ID NO: 1) comprises L1 region from positions 5559 to 7154, genomic DNA sequence of HPV-52 (GenBank accession number: NC_{—}001592; SEQ ID NO: 2) comprises L1 region from positions 5565 to 7154, genomic DNA sequence of HPV-58 (GenBank accession number: NC_{—}001443; SEQ ID NO: 3) comprises L1 region from positions 5565 to 7139, genomic DNA sequence of HPV-18 (GenBank accession number: NC_{—}001357) comprises L1 region from positions 5430 to 7136, genomic DNA sequence of HPV-31 (GenBank accession number: J04353) comprises L1 region from positions 5552 to 7066, genomic DNA sequence of HPV-33 (GenBank accession number: NC_{—}001528) comprises L1 region from positions 5594 to 7093, and genomic DNA sequence of HPV-35 (GenBank accession number: M74117) comprises L1 region from positions 5574 to 7091.

As used herein, “severe dysplasia and lesion in more advanced stages” are lesions classified as “severe dysplasia”, “intraepithelial cancer”, “microinvasive squamous cancer” and “invasive squamous cancer” based on the classification according to “General Rules for Clinical and Pathological Study of Uterine Cervical Cancer in Japan 1997” by Japan Society of Obstetrics and Gynecology.

The subjects who are diagnosed as these lesions need to undergo treatment such as surgical operations; thus it is clinically important to determine whether or not it is severe dysplasia or lesion in more advanced stages.

The lesions in less advanced stages than “severe dysplasia” are classified to “normal in epithelium”, “mild dysplasia” and “moderate dysplasia”. Most of the subjects who are diagnosed as these lesions undergo follow-up without any treatment.

As used herein, “abnormal cells” denote atypical cells and cancer cells. Atypical cells denote the cells which are not cancer cells but are recognized with nuclear abnormalities such as nuclear enlargement, increase in chromatin, irregular nucleus and the like.

In the present invention, a sample obtained from uterine cervix of a subject is not specifically limited so long as it contains DNA comprised in swabs collected by smearing uterine cervix or tissues collected from uterine cervix. Preferably, it is a processed sample obtained by treating a swab, tissue or a paraffin block of tissue with an appropriate treatment solution. The treatment solution is preferably a buffer containing a surfactant. The processed sample is more preferably obtained by suspending a swab, tissue or a paraffin block of tissue in the treatment solution and homogenizing the suspension.

Tissue may be collected from uterine cervix by well-known methods in the art such as an excision by surgical operations (conization, total hysterectomy etc.), biopsy carried out under the observation with colposcopy.

DNA contained in the sample may preferably be purified, although it is not necessary. The purification of DNA in the sample may be carried out by well-known methods in the art such as ethanol precipitation, phenol/chloroform extraction, use of commercially available nucleic acid purification kits.

The detection of methylation of CpG(s) which is(are) expected to be methylated in L1 region of HPV genomic DNA in the sample may be carried out by methods well-known in the art. Such methods include Bisulfite Sequencing based on the procedures of treating DNA with bisulfite to transform unmethylated cytosine(s) to uracil(s), amplifying DNA in a target region by PCR and sequencing the amplified DNA region (see, for example, T. Turan et al., “Methylation of human papillomavirus-18 L1 gene: A biomarker of neoplastic progression?” Virology 349 (2006) p. 175-183), Methylation-Specific PCR (James G. HERMAN et al., Methylation-specific PCR: A novel PCR assay for methylation status of CpG islands, Proc. Natl. Acad. Sci. USA, Vol. 93, pp. 9821-9826, September 1996), and a method described in WO 2006/132022 based on the oxidization of methylated cytosine with a guide probe.

According to Bisulfite Sequencing, DNA in a sample is reacted with bisulfite such as sodium bisulfite or potassium bisulfite to transform unmethylated cytosine(s) in DNA to uracil(s). Methylated cytosine(s) is(are) not transformed to uracil(s).

The concentration of bisulfite in the transformation of unmethylated cytosine is not specifically limited so long as unmethylated cytosine(s) in DNA in a sample can be sufficiently transformed. More specifically, the concentration of bisulfite is 1M or more, preferably 1M to 15M and more preferably 3M to 10M. When the final concentration of sodium bisulfite added in a sample is 4M, unmethylated cytosine(s) can be transformed to uracil(s) with the incubation at 50° C. to 80° C. for 10 to 90 minutes. When bisulfite is used at lower concentration, the incubation time and temperature may be appropriately changed to such extent that unmethylated cytosine(s) are sufficiently transformed.

Next, DNA which has been reacted with bisulfite is amplified with the primer set of the present invention (see below) by a nucleic acid amplification method. The nucleic acid amplification method is not specifically limited and is a well-known nucleic acid amplification method such as PCR or LAMP. The conditions for nucleic acid amplification method may be appropriately selected by a skilled person in the art according to the method to be used, base sequence of the DNA region to be amplified, base sequence of primers and the like.

The primer set of the present invention can amplify a region which comprises at least one CpG existing within 80% from 5′-terminal among all CpGs in L1 region of HPV genomic DNA and has been treated with bisulfite. Preferably, it can amplify the region which does not comprise CpG(s) existing within 20% from 3′-terminal and has been treated with bisulfite.

The base sequences of primers comprised in the present primer set may be such that they can hybridize with a partial base sequence of DNA comprising the analytical region and having been treated with bisulfite and they can initiate amplification of DNA corresponding to the above region in the nucleic acid amplification method.

When HPV to be analyzed is HPV-16, the primer set preferably amplifies, in a nucleic acid amplification method, a region which comprises at least one CpG among the 1^{st} to 15^{th} CpGs from 5′-terminal of L1 region of HPV-16 and does not comprise the 16^{th} to 19^{th} CpGs and has been treated with bisulfite. Specific primer sets are shown below.

The primer set consisting of the primers having the sequences SEQ ID NOs: 8 and 9 amplifies, in a nucleic acid amplification method, a region which comprises the 11^{th} to 15^{th} CpGs from 5′-terminal of L1 region of HPV-16 and has been treated with bisulfite:

| | |
|---|---|
| SEQ ID NO: 8: | AATAGGGTTGGTATTGTTGGTGAAAAT |
| SEQ ID NO: 9: | TTCCAATCCTCCAAAATAATAAAATTCATA |

The primer set consisting of the primers having the sequences SEQ ID NOs: 24 and 25 amplifies, in a nucleic acid amplification method, a region which comprises the 1st to 8th CpGs from 5′-terminal of L1 region of HPV-16 and has been treated with bisulfite:

SEQ ID NO: 24:
TTGTTGATGTAGGTGATTTTTATTTATATTTTAGTT
SEQ ID NO: 25:
CCACTAATACCCACACCTAATAACTAACC

The primer set consisting of the primers having the sequences SEQ ID NOs: 32 and 33 amplifies, in a nucleic acid amplification method, a region which comprises the 1^{st} to 6^{th} CpGs from 5′-terminal of L1 region of HPV-16 and has been treated with bisulfite:

| | |
|---|---|
| SEQ ID NO: 32: | GATGTAGGTGATTTTTATTTATATTTTAGTT |
| SEQ ID NO: 33: | ATCCAACTACAAATAATCTAAATATTC |

When HPV to be analyzed is HPV-18, the primer set preferably amplifies, in a nucleic acid amplification method, a region which comprises at least one CpG among the 1^{st} to 25^{th} CpGs from 5′-terminal of L1 region of HPV-18 and does not comprise the 26^{th} to 32^{nd} CpGs and has been treated with bisulfite. Specific primer set is shown below.

The primer set consisting of the primers having the sequences SEQ ID NOs: 38 and 39 amplifies, in a nucleic acid amplification method, a region which comprises the 9^{th} to 16^{th} CpGs from 5′-terminal of L1 region of HPV-18 and has been treated with the bisulfite:

| | |
|---|---|
| SEQ ID NO: 38: | GTTATTTGATTTAAATAAATTTGGTTTATTTGA |
| SEQ ID NO: 39: | TCCATAACACCATATCCAATATCTACC |

When HPV to be analyzed is HPV-31, the primer set preferably amplifies, in a nucleic acid amplification method, a region which comprises at least one CpG among the 1^{st} to 17^{th} CpGs from 5′-terminal of L1 region of HPV-31 and does not comprise the 18^{th} to 22^{nd} CpGs and has been treated with bisulfite. Specific primer set is shown below.

The primer set consisting of the primers having the sequences SEQ ID NOs: 36 and 37 amplifies, in a nucleic acid amplification method, a region which comprises the 11^{th} to 17^{th} CpGs from 5′-terminal of L1 region of HPV-31 and has been treated with bisulfite:

| | |
|---|---|
| SEQ ID NO: 36: | GGTGATTGTTTTTTATTAGAATTAAAAA |
| SEQ ID NO: 37: | AATACCATTATTATATCCCTAAACAC |

When HPV to be analyzed is HPV-33, the primer set preferably amplifies, in a nucleic acid amplification method, a region which comprises at least one CpG among the 1^{st} to 16^{th} CpGs from 5′-terminal of L1 region of HPV-33 and does not comprise the 17^{th} to 21^{st} CpGs and has been treated with bisulfite. Specific primer set is shown below.

The primer set consisting of the primers having the sequences SEQ ID NOs: 40 and 41 amplifies, in a nucleic acid amplification method, a region which comprises the 4^{th} to 9^{th} CpGs from 5′-terminal of L1 region of HPV-33 and has been treated with bisulfite:

| | |
|---|---|
| SEQ ID NO: 40: | TGGTAGTTTTAGATTTTTTGTTGTT |
| SEQ ID NO: 41: | CTTTACCCCAATATTCCCCTA |

When HPV to be analyzed is HPV-35, the primer set preferably amplifies, in a nucleic acid amplification method, a region which comprises at least one CpG among the 1^{st} to 13^{th} CpGs from 5′-terminal of L1 region of HPV-35 and does not comprise the 14^{th} to 17^{th} CpGs and has been treated with bisulfite. Specific primer set is shown below.

The primer set consisting of the primers having the sequences SEQ ID NOs: 42 and 43 amplifies, in a nucleic acid amplification method, a region which comprises the 8^{th} to 13^{th} CpGs from 5′-terminal of L1 region of HPV-35 and has been treated with bisulfite:

| | |
|---|---|
| SEQ ID NO: 42: | TTTTTAGTGGTTTTATGGTAATTTT |
| SEQ ID NO: 43: | AAATTTTTAACAAATTACAACCTATAATA |

When HPV to be analyzed is HPV-52, the primer set preferably amplifies, in a nucleic acid amplification method, a region which comprises at least one CpG among the 1^{st} to 17^{th} CpGs from 5′-terminal of L1 region of HPV-52 and does not comprise the 18^{th} to 22^{nd} CpGs and has been treated with bisulfite. Specific primer sets are shown below.

The primer set consisting of the primers having the sequences SEQ ID NOs: 12 and 13 amplifies, in a nucleic acid amplification method, a region which comprises the 1^{st} to 9^{th} CpGs from 5′-terminal of L1 region of HPV-52 and has been treated with bisulfite:

| | |
|---|---|
| SEQ ID NO: 12: | TTTATTTATATTTATTATTGTTGATGGTAT |
| SEQ ID NO: 13: | TAAAAAACCAAATTTATTAAAATCC |

The primer set consisting of the primers having the sequences SEQ ID NOs: 26 and 27 amplifies, in a nucleic acid amplification method, a region which comprises the 13^{th} to 18^{th} CpGs from 5′-terminal of L1 region of HPV-52 and has been treated with bisulfite:

| | |
|---|---|
| SEQ ID NO: 26: | TTTAGGTGATTTTGTGTTAGGT |
| SEQ ID NO: 27: | TCCTCTAAAATAATAACATCCATCT |

The primer set consisting of the primers having the sequences SEQ ID NOs: 28 and 29 amplifies, in a nucleic acid amplification method, a region which comprises the 10^{th} to 15^{th} CpGs from 5′-terminal of L1 region of HPV-52 and has been treated with bisulfite:

| | |
|---|---|
| SEQ ID NO: 28: | TTGGTTGTATGGATTTTAATATTT |
| SEQ ID NO: 29: | AACAAACAACTAATTACCCCA |

When HPV to be analyzed is HPV-58, the primer set preferably amplifies, in a nucleic acid amplification method, a region which comprises at least one CpG among the 1^{st} to 19^{th} CpGs from 5′-terminal of L1 region of HPV-58 and does not comprise the 20^{th} to 25^{th} CpGs and has been treated with bisulfite. Specific primer sets are shown below.

The primer set consisting of the primers having the sequences SEQ ID NOs: 18 and 19 amplifies, in a nucleic acid amplification method, a region which comprises the 1^{st} to 9^{th} CpGs from 5′-terminal of L1 region of HPV-58 and has been treated with bisulfite:

| | |
|---|---|
| SEQ ID NO: 18: | ATGGTGTTGATTTTATGTTGTATT |
| SEQ ID NO: 19: | AACTATCCCCTACCTATTTCAAAAC |

The primer set consisting of the primers having the sequences SEQ ID NOs: 30 and 31 amplifies, in a nucleic acid amplification method, a region which comprises the 12^{th} to 19^{th} CpGs from 5′-terminal of L1 region of HPV-58 and has been treated with bisulfite:

| | |
|---|---|
| SEQ ID NO: 30: | TGGTTAGTGAATTTTATGGGG |
| SEQ ID NO: 31: | TTACAAAACTAAAAAACAAACTATAAATCA |

Then, the DNA region amplified by a nucleic acid amplification method is sequenced. When the base at the position which is expected to be cytosine according to the sequence information obtained from the public database described above is transformed to uracil (thymine), this base can be determined to be unmethylated cytosine.

When the base at the position which is expected to be cytosine is found to be cytosine in the sequencing, it can be determined to be methylated cytosine.

The sequencing can be carried out by well-known methods in the art, for example, by using a DNA sequencer.

By detecting the number of methylated CpG(s) in L1 region of HPV genomic DNA which has been analyzed according to the above method, the frequency of methylation can be measured. The frequency of methylation can also be measured by dividing the number of methylated CpG(s) by the number of all CpGs in the region.

Based on the frequency of methylation measured as above, the determination on whether or not a sample from a subject contains abnormal cells originated from severe dysplasia or lesion in more advanced stages of uterine cervix or the prediction on whether or not uterine cervical tissue of a subject progresses to severe dysplasia or lesion in more advanced stages is carried out. Such detection and prediction can be carried out by, for example, comparing the frequency of methylation measured and a predetermined threshold.

More specifically, when the frequency of methylation measured for a sample from a subject is higher than the predetermined threshold, the sample can be judged to contain abnormal cells. This is synonymous with judging that the sample does not contain abnormal cells when the frequency of methylation is at or lower than the threshold.

Similarly, when he frequency of methylation measured for a sample from a subject is higher than the predetermined threshold, it can be predicted that uterine cervical lesion of the subject may progress to severe dysplasia or lesion in more advanced stages, even when lesion of the subject has been diagnosed as in less advanced stages than “severe dysplasia” according to the conventional diagnosis methods. This is synonymous with that predicting the lesion may not progress to severe dysplasia or lesion in more advanced stages when the frequency of methylation is at or lower than the threshold.

When the frequency of methylation is at or lower than the threshold, it is also possible to predict that uterine cervical lesion of the subject may disappear.

The threshold to be used for the determination of presence of absence of abnormal cells described above can be decided based on the known severity of lesions from patients and the frequency of methylation in L1 region of HPV genomic DNA in the samples from these patients.

The threshold may be decided as specifically described below. For determination of a threshold, a sample is used which is obtained from uterine cervix of a subject whose severity of lesion has been known according to the method other than the present method such as histological diagnosis. The frequency of methylation in L1 region of HPV genomic DNA for this sample is measured. The result of the measurement is then compared to the severity of lesion determined by the method other than the present method. Then, the threshold can be the value of the frequency of methylation that can most clearly differentiate the frequency of methylation for a sample of a subject who has been determined to have severe dysplasia or lesion in more advanced stages according to the method other than the present method and the frequency of methylation for a sample of a subject who has been determined to have lesion in less advanced stages than severe dysplasia.

For example, the analytical region is set to a region which contains 10 CpGs in total in L1 region. When, for this region, more than 2 methylated CpGs are detected for a sample of a subject who has been determined to have severe dysplasia or lesion in more advanced stages according to histological diagnosis and 1 or less methylated CpG is detected for a sample from a subject who has been determined to have lesion in less advanced stages than severe dysplasia according to histological diagnosis, a threshold can be 2. Thus, when the number of methylated CpGs in a sample from a subject is measured as 2 or more, it can be determined that the sample contains abnormal cells originated from severe dysplasia or lesion in more advanced stages.

When the frequency of methylation is calculated according to the above formula I, a threshold can be 20% in the above example. Thus, when the ratio of methylated CpGs relative to the total CpGs in a sample from a subject is measured as 20% or more, it can be determined that the sample contains abnormal cells originated from severe dysplasia or lesion in more advanced stages.

The threshold to be used for the determination of presence or absence of abnormal cells can also be used for the prediction on progression of lesion. The threshold can also be determined based on the information on progression of lesion of a patient whose progression is known and the frequency of methylation in L1 region of HPV genomic DNA in a sample from the patient collected during a follow-up of progression.

The threshold in this case may be decided as specifically described below. For determination of a threshold, a sample is used which is obtained from a subject, during a follow-up of progression, whose progression information is known whether lesion has been disappeared or progressed to severe dysplasia or lesion in more advanced stages. The frequency of methylation in L1 region of HPV genomic DNA for this sample is measured. The result of the measurement is compared to the progression information. Then, the threshold can be the value of the frequency of methylation which can most clearly differentiate the frequency of methylation for a sample of a subject whose progression information corresponds to the progression to severe dysplasia or lesion in more advanced stages and the frequency of methylation for a sample of a subject whose progression information corresponds to the disappearance of lesion.

### EXAMPLES

The present invention is illustrated in more detail by the following Examples which do not intend to limit the present invention.

### Example 1

1. Samples

A paraffin block of uterine cervical tissue which was surgically obtained from a subject was sectioned and classified to “mild dysplasia (CIN1)”, “moderate dysplasia (CIN2) or “severe dysplasia (CIN3 or more)” by histological diagnosis (hematoxylin-eosin staining).

To three paraffin block sections with 10 μm thick from the same subject was added 1 ml xylene and mixed. The mixture was centrifuged and then the supernatant was discarded. The precipitate was added with 1 mL of 100% ethanol to wash. This washing with ethanol was repeated once more. The washed precipitate was incubated at 37° C. for 10 minutes to evaporate ethanol. Thus obtained precipitate is hereinafter referred to as an “operation sample”.

A paraffin block of a sample obtained from a subject by scraping a part of uterine cervical tissue under observation with colposcopy was sectioned and classified for the severity of lesion by histological diagnosis as described above.

To three paraffin block sections with 10 μm thick from the same subject was added 1 ml xylene and mixed. The mixture was centrifuged and then the supernatant was discarded. The precipitate was added with 1 mL of 100% ethanol to wash. This washing with ethanol was repeated once more. The washed precipitate was incubated at 37° C. for 10 minutes to evaporate ethanol. Thus obtained precipitate is hereinafter referred to as a “biopsy sample”.

2. Bisulfite Sequencing

2-1. Bisulfite Treatment

To the above operation sample or biopsy sample was added 500 μl of the solution containing 1% (w/v) SDS and 0.1 M NaOH. The obtained mixture was incubated at 100° C. for 20 minutes. The incubated mixture was centrifuged at 4° C. and the supernatant was collected.

To the obtained supernatant was added 500 μl of 10M bisulfite solution and mixed. The obtained mixture was incubated at 80° C. for 40 minutes to carry out a bisulfite treatment. Nucleic acid contained in the bisulfite treated solution was purified with a nucleic acid purification kit (QIAquick PCR purification kit, QIAGEN). Sodium hydroxide was added to the obtained nucleic acid to the final concentration of 0.3M. The obtained mixture was incubated at room temperature for 5 minutes. Thus obtained product was purified on a spin column for nucleic acid purification (Micro Spin S-300 HR Columns, GE Healthcare) to obtain 50 μl of a bisulfite treated template DNA sample.

2-2. PCR Reaction

PCR was carried out on nucleic acid in the bisulfite treated template DNA sample with primer sets shown in the following Table 1.

These primer sets have sequences suitable for amplifying partial regions of HPV genomic DNAs shown as “Amplified region” in Table 1.

TABLE 1

| | SEQ | | | | |
|---|---|---|---|---|---|
| Target | ID | | Amplified | Amplified | |
| HPV | NO: | Name | region | size | Sequence |
| HPV-16 | 4 | 2F(16) | LCR | 436 bp | TTAATATTTATTAATTGTGTTGTGGTTATTTATTG |
| | 5 | 2R(16) | | | TAACCTTAAAAATTTAAACCTTATACCAAATATAC |
| | 24 | 5F(16) | L1 | 439 bp | TTGTTGATGTAGGTGATTTTTATTTATATTTTAG |
| | | | | | TT |
| | 25 | 5R(16) | Anterior* | | CCACTAATACCCACACCTAATAACTAACC |
| | 6 | 7F(16) | L1-LCR | 440 bp | AGTAGGATTGAAGGTTAAATTAAAATTTAT |
| | 7 | 7R(16) | | | AACACATTTTATACCAAAAAACATACAACC |
| | 8 | 6F(16) | L1 | 424 bp | AATAGGGTTGGTATTGTTGGTGAAAAT |
| | 9 | 6R(16) | | | TTCCAATCCTCCAAAATAATAAAATTCATA |
| HPV-52 | 10 | 1F-2 | LCR | 455 bp | GTATTATTTTGTATTATTTATTATTTTAAAT |
| | | (52) | | | |
| | 11 | 1R-2(52) | | | CCCTATTTTTTACTAATATAAAATTATAATTTA |
| | 26 | 5F(52) | L1 | 408 bp | TTTAGGTGATTTTGTGTTAGGT |
| | 27 | 5R(52) | Middle* | | TCCTCTAAAATAATAACATCCATCT |
| | 28 | 4F(52) | L1 | 385 bp | TTGGTTGTATGGATTTTAATATTT |
| | 29 | 4R(52) | Middle* | | AACAAACAACTAATTACCCCA |
| | 12 | 2F(52) | L1 | 367 bp | TTTATTTATATTTATTATTGTTGATGGTAT |
| | 13 | 2R(52) | Anterior | | TAAAAAACCAAATTTATTAAAATCC |
| | 14 | 3F(52) | L1 | 299 bp | ATTTTAGAGGATTGGTAATTTGG |
| | 15 | 3R(52) | Posterior* | | AACCTTTTTCTTCTTTATAAAAATAC |
| HPV-58 | 16 | 1F(58) | LCR | 471 bp | TTTTATTTTTATTTTGTGTATGTAAT |
| | 17 | 1R-2(58) | | | TAATCCTACAATAACCTACCAAAAA |
| | 30 | 5F(58) | L1 | 427 bp | TGGTTAGTGAATTTTATGGGG |
| | 31 | 5R-2(58) | Middle* | | TTACAAAACTAAAAAACAAACTATAAATCA |
| | 18 | 2F(58) | L1 | 415 bp | ATGGTGTTGATTTTATGTTGTATT |
| | 19 | 2R(58) | Anterior* | | AACTATCCCCTACCTATTTCAAAAC |
| | 20 | 3F(58) | L1 | 344 bp | TTAATATTTTGGAGGATTGGTAAT |
| | 21 | 3R(58) | Posterior* | | ATATAATAAAATAATATAAATACCACAACA |
| | 22 | 4F(58) | L1-LCR | 489 bp | AATTAGGTTTTAAAGTAAAGTTTAGATTA |
| | 23 | 4R(58) | | | TTATTTAAATTATAATTTAAAAAAAACAC |

*“L1 Anterior” means a region proximal to 5′-terminal of L1 region, “L1 Posterior” mans a region proximal to 3′-terminal of L1 region and “L1 Middle” means a central region of L1 region.

The composition of PCR reaction solution is as shown below. DNA polymerase used was Ex Taq® Polymerase (TaKaRa Bio Inc.).

| | | |
|---|---|---|
| 10 x buffer | 1.5 | μl |
| 2.5 mM dNTP | 1.2 | μl |
| Forward primer (10 μM) | 0.6 | μl |
| Reverse primer (10 μM) | 0.6 | μl |
| DNA polymerase | 0.075 | μl |
| Water | 9.025 | μl |
| Template DNA sample | 2 | μl |
| Total | 15 | μl |

The following Table 2 shows the PCR conditions.

TABLE 2

| | HPV-16/HPV-52 | | | HPV-58 | | |
|---|---|---|---|---|---|---|
| | Temp. (° C.) | Time | | Temp. (° C.) | Time | |
| Hot start | 95 | 4.5 | min. | 95 | 4.5 | min. |
| Denaturing | 95 | 30 | sec. | 95 | 30 | sec. |
| Annealing | 52 | 30 | sec. | 54 | 30 | sec. |
| Extension | 72 | 40 | sec. | 72 | 40 | sec. |

| | | |
|---|---|---|
| Cycles | 40 or 45 | 40 or 45 |

2-3. TA Cloning and DNA Sequencing

The PCR amplified product was incorporated into a vector provided with TA Cloning Kit (Invitrogen). The obtained construct was used for the transformation of Escherichia coli TOP 10. The transformed E. coli was cultured overnight on a LB agar medium (1% (w/v) trypton, 0.5% (w/v) yeast extract, 1% (w/v) sodium chloride, and 1.5% (w/v) agar) at 37° C.

Among thus obtained E. coli colonies, bacteria harboring the vector containing the PCR product was selected by colony-PCR method and incubated overnight in LB liquid medium at 37° C.

A glycerol stock of the cultured E. coli was prepared and DNA sequencing of the vector contained in bacteria was carried out at TaKaRa Bio Inc.

Cytosine was determined to be methylated when it was expected to be cytosine based on the sequences SEQ ID NOs: 1 to 3 and it was determined to be cytosine according to the above sequencing. Cytosine was determined to be not methylated when it was expected to be cytosine based on the sequences SEQ ID NOs: 1 to 3 but it was determined to be transformed to uracil (thymine).

3. Results

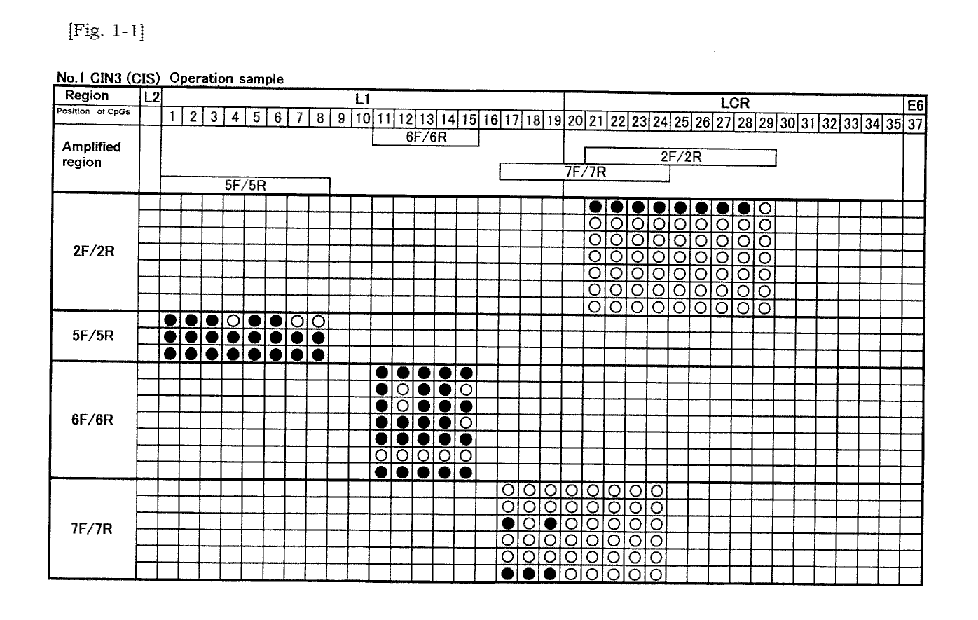

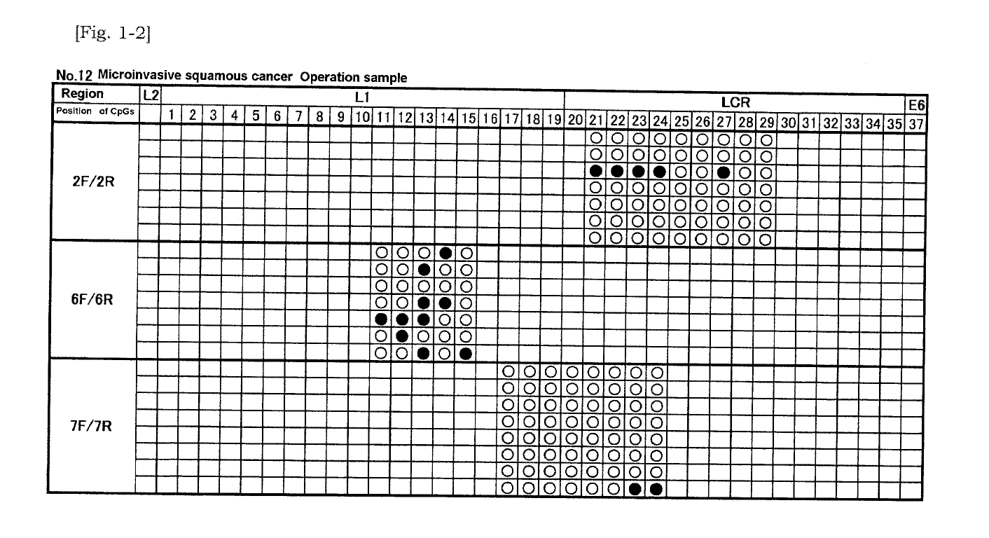

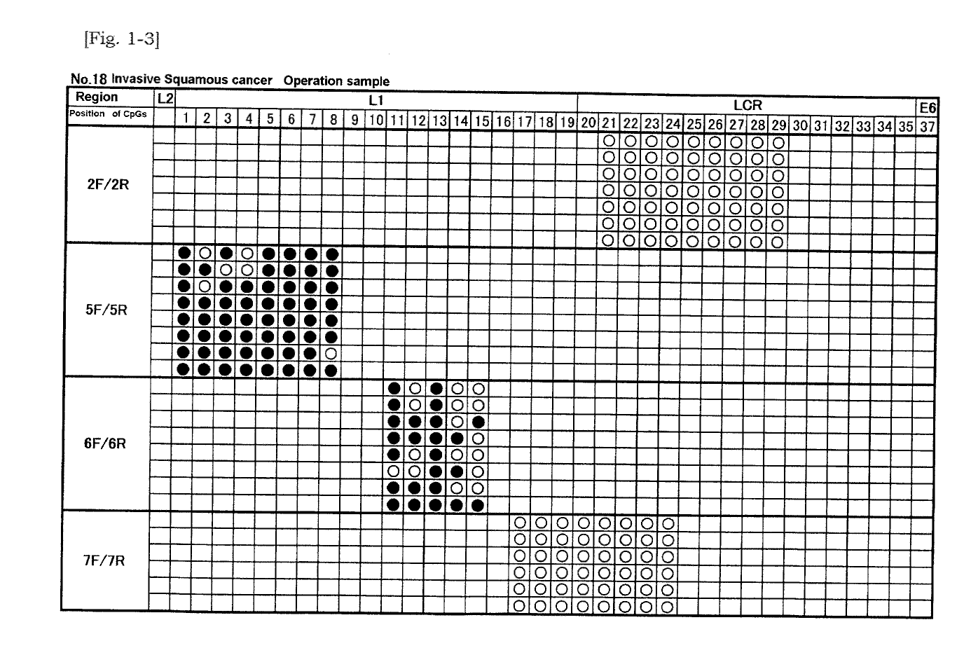

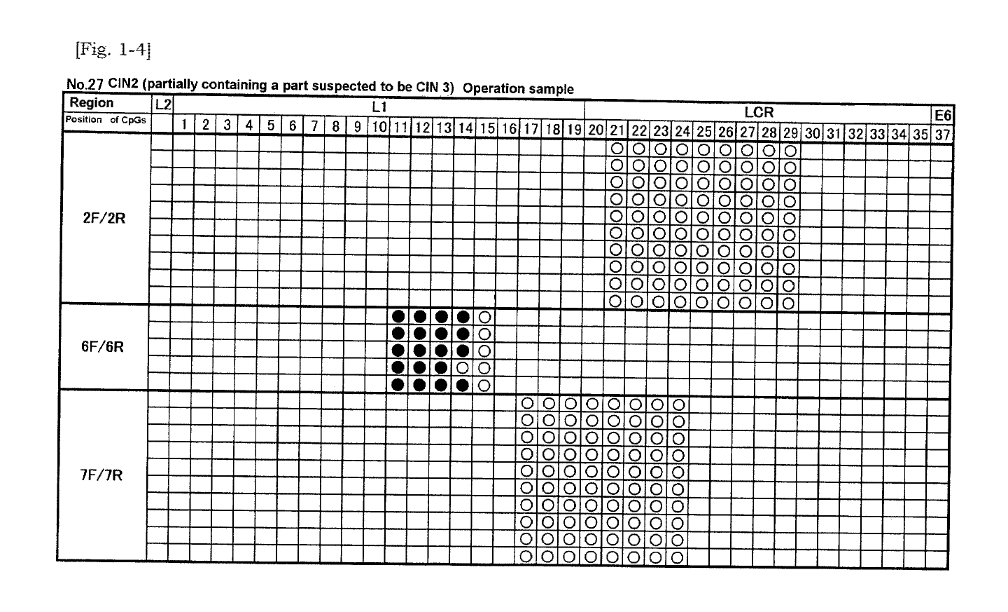

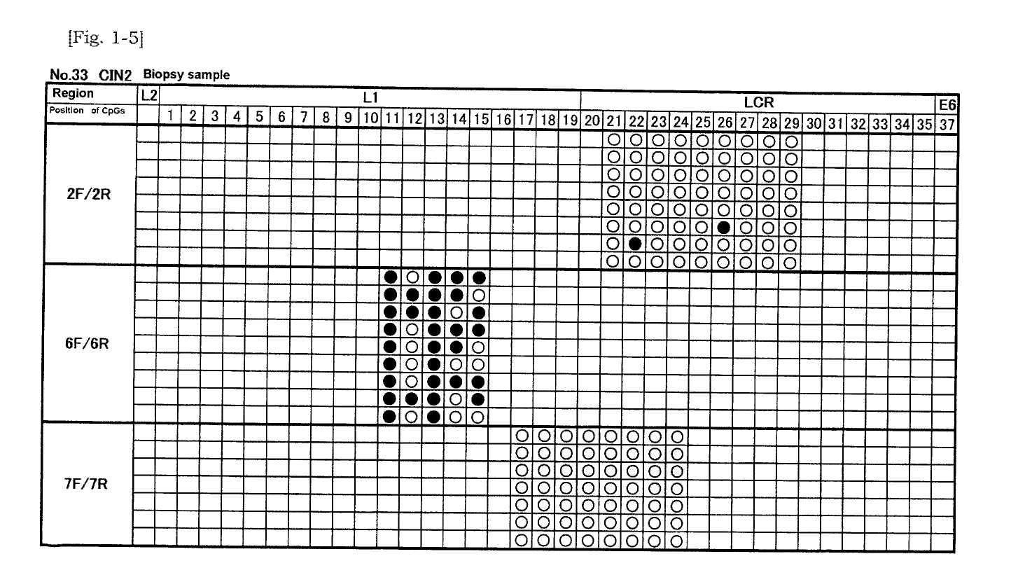

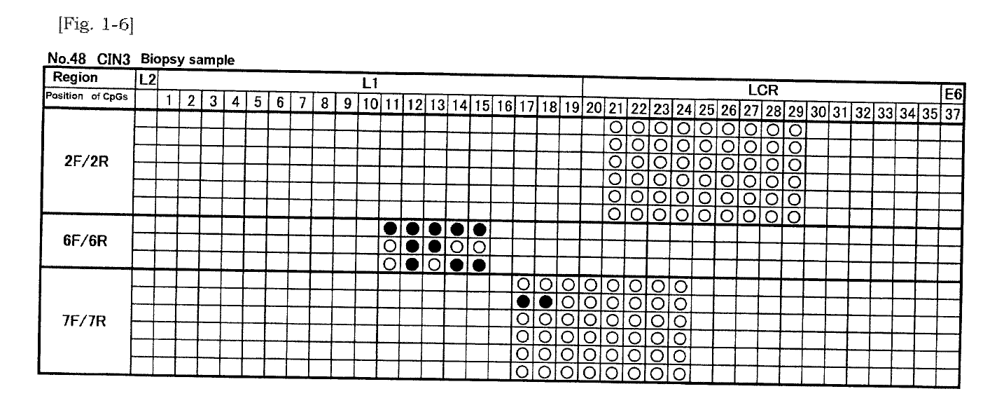

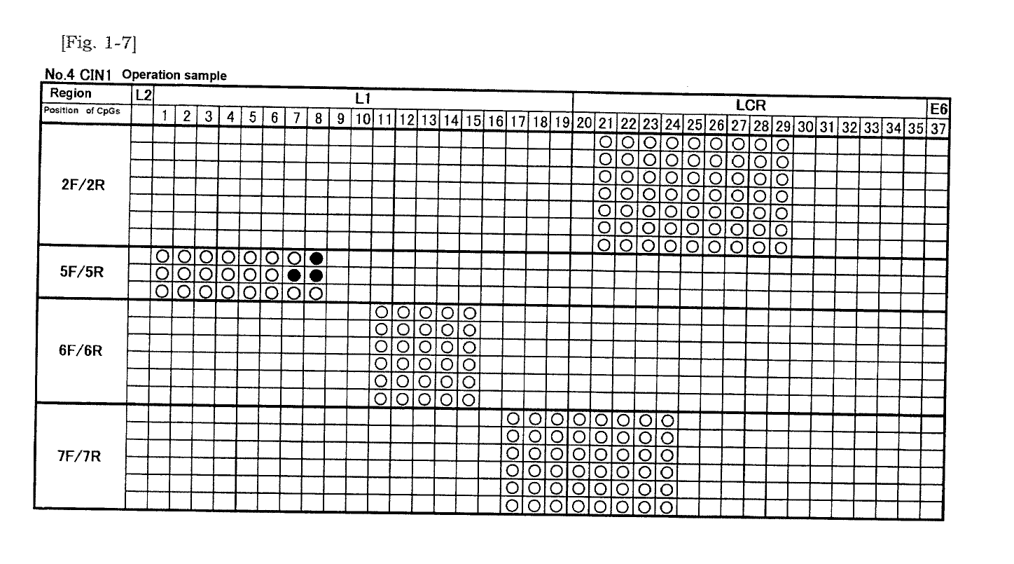

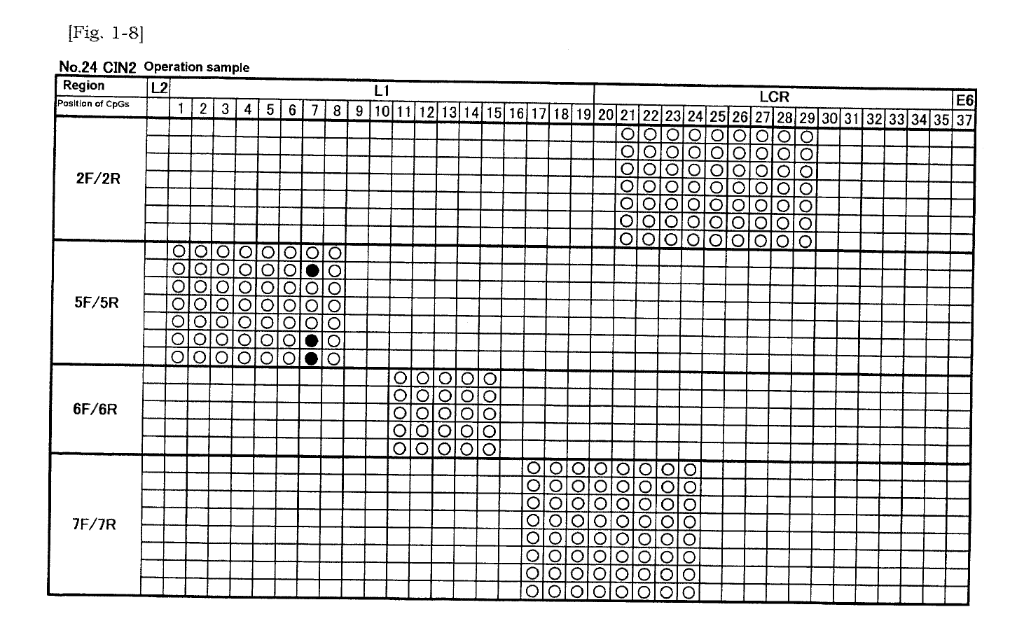

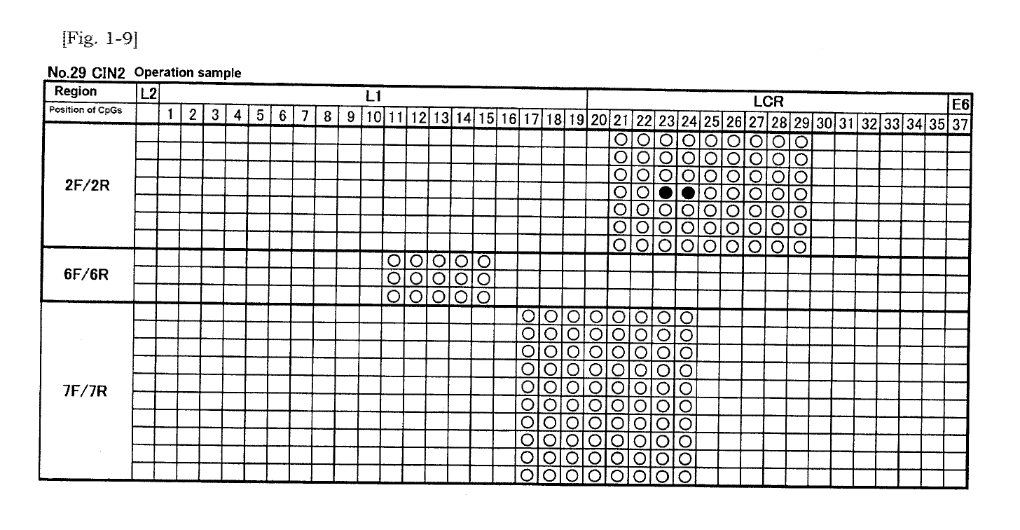

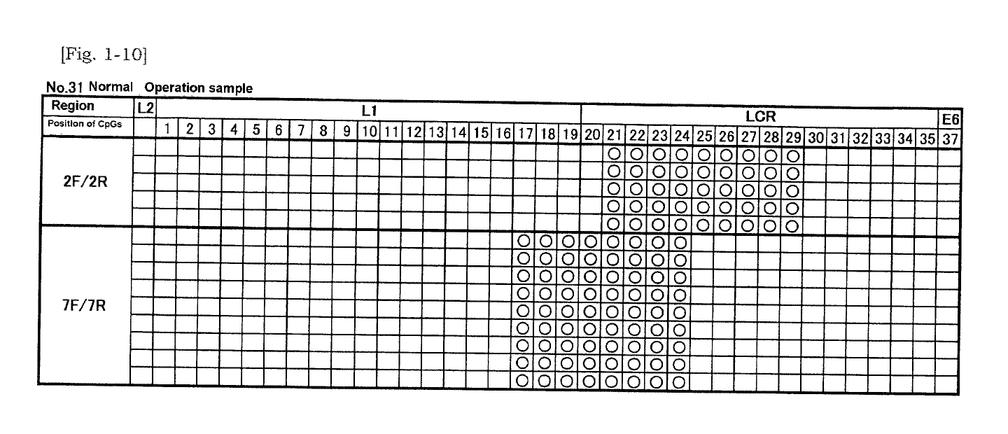

As described above, methylated CpGs were detected in L1 or LCR region of HPV genomic DNA in samples. Some of the results are shown in FIGS. 1 to 3.

FIG. 1 shows the results of detection of methylated CpGs in L1 region or LCR of HPV-16 genomic DNA.

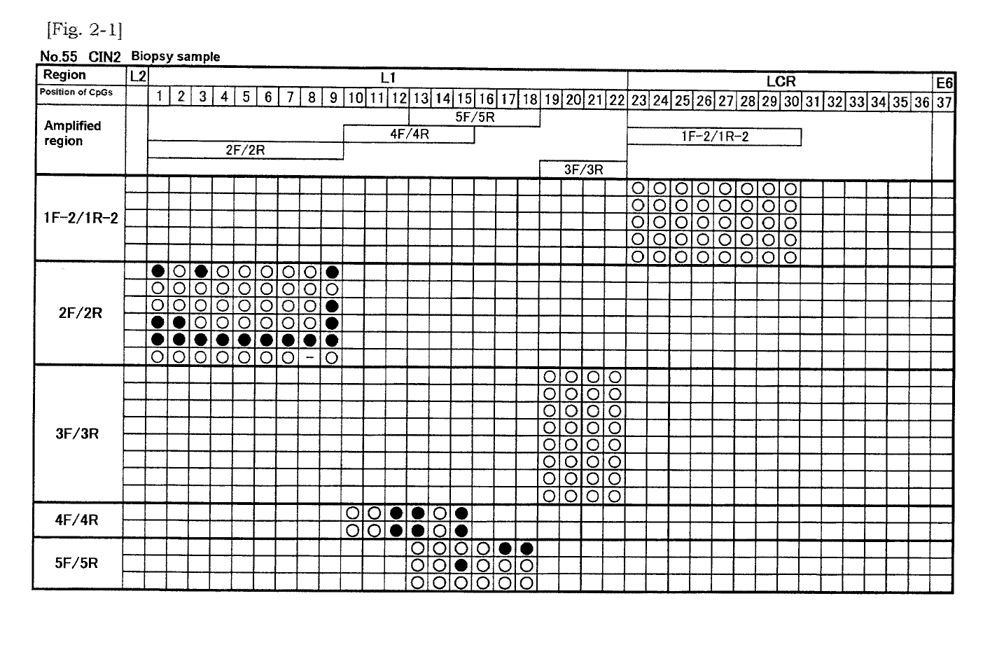

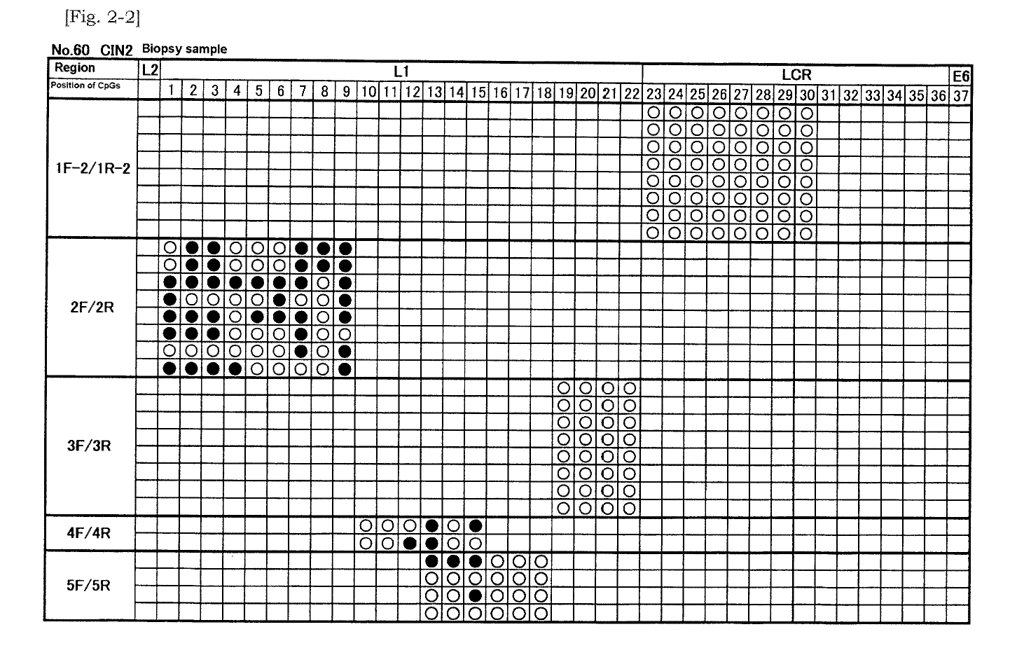

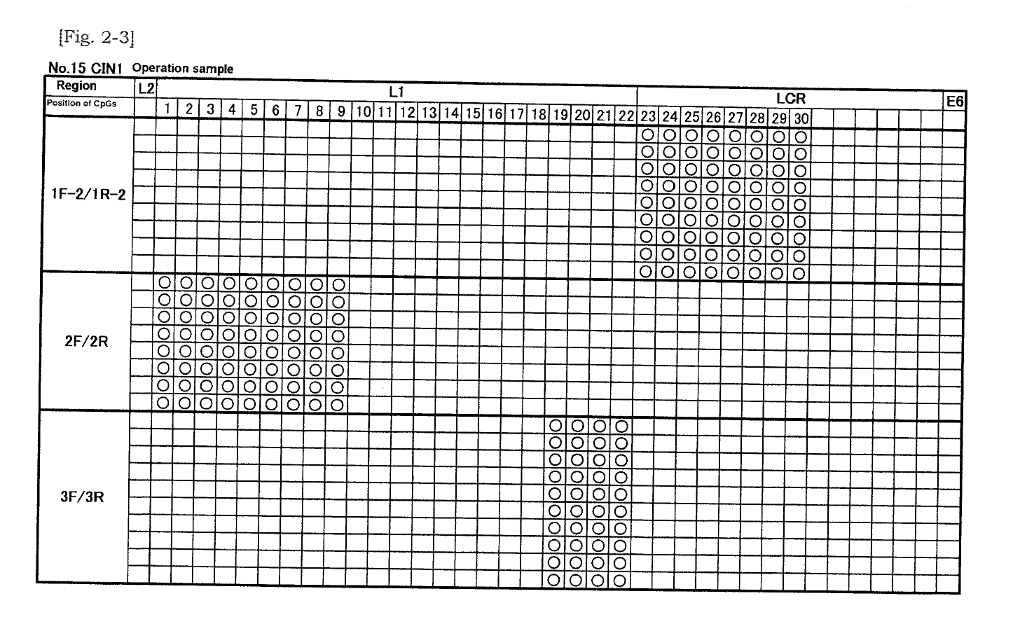

FIG. 2 shows the results of detection of methylated CpGs in L1 region or LCR of HPV-52 genomic DNA.

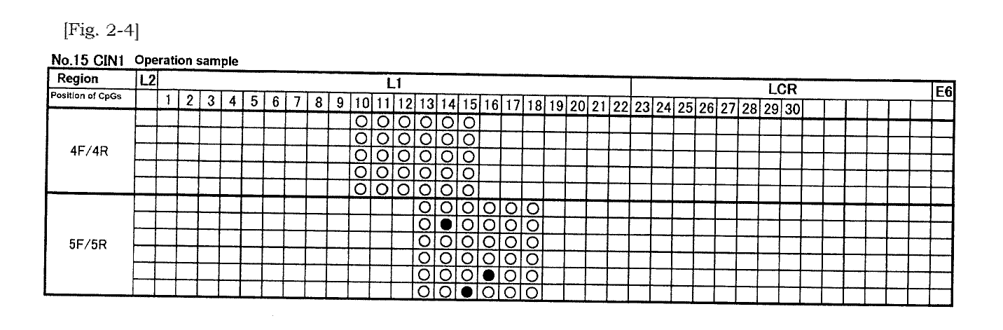

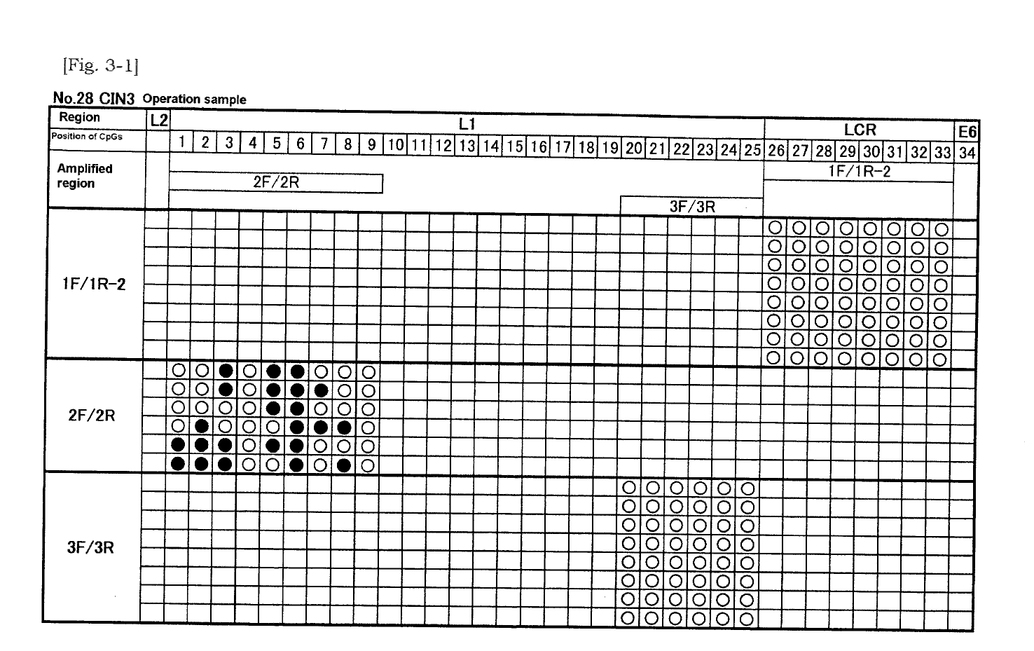

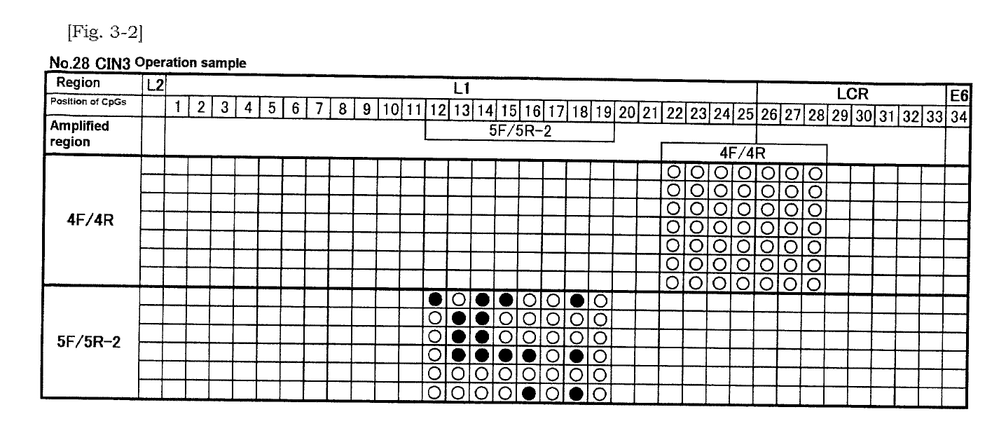

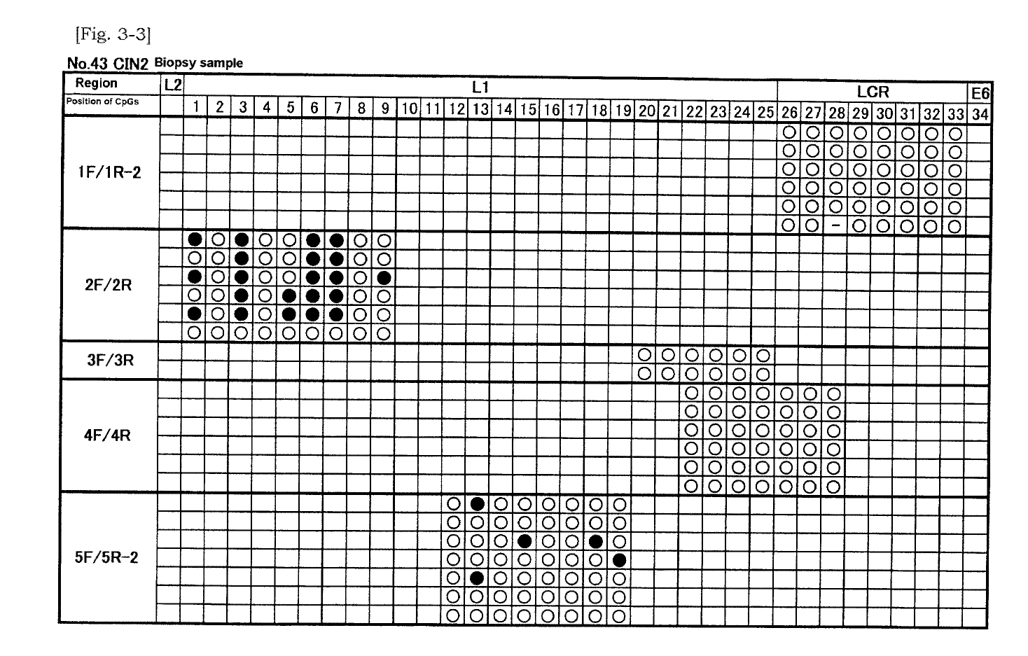

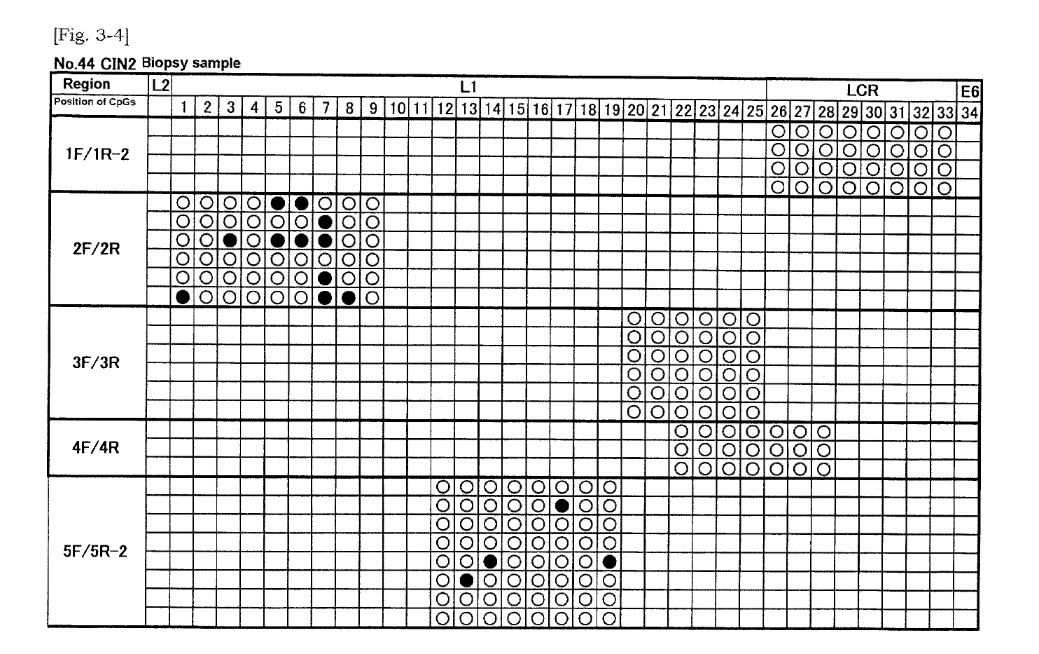

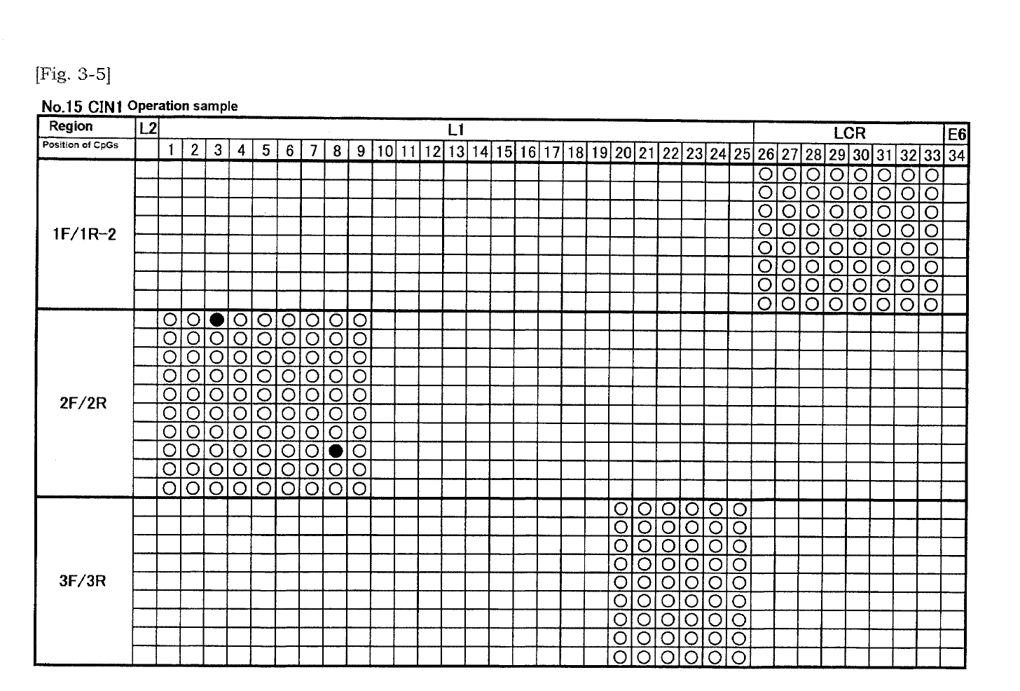

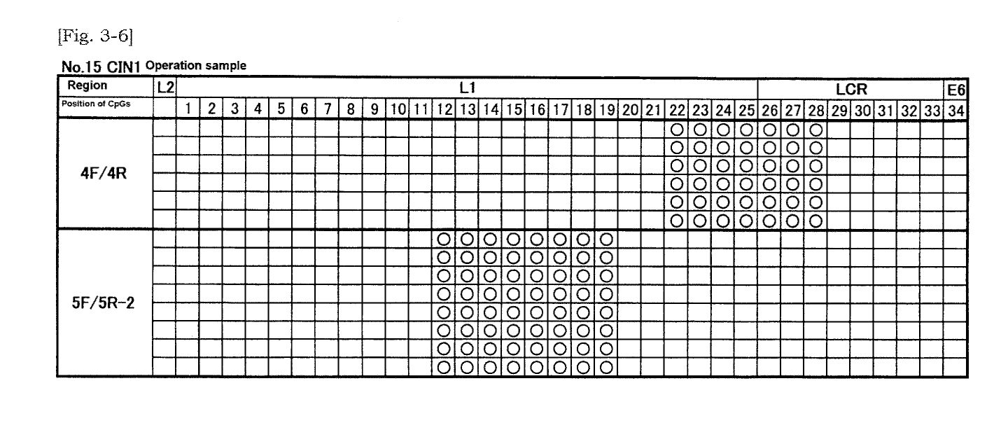

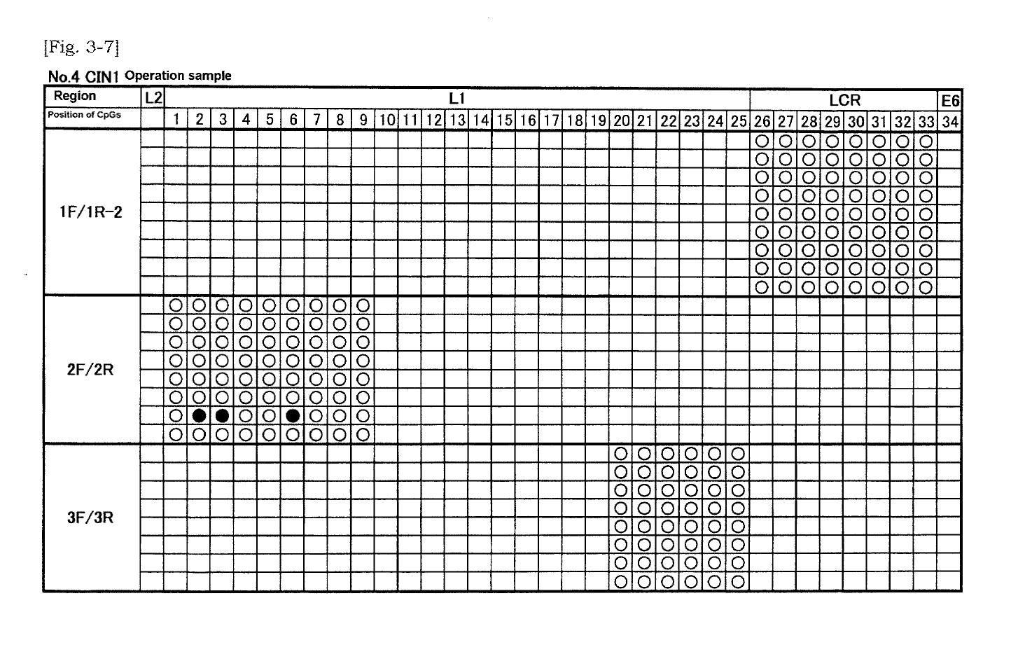

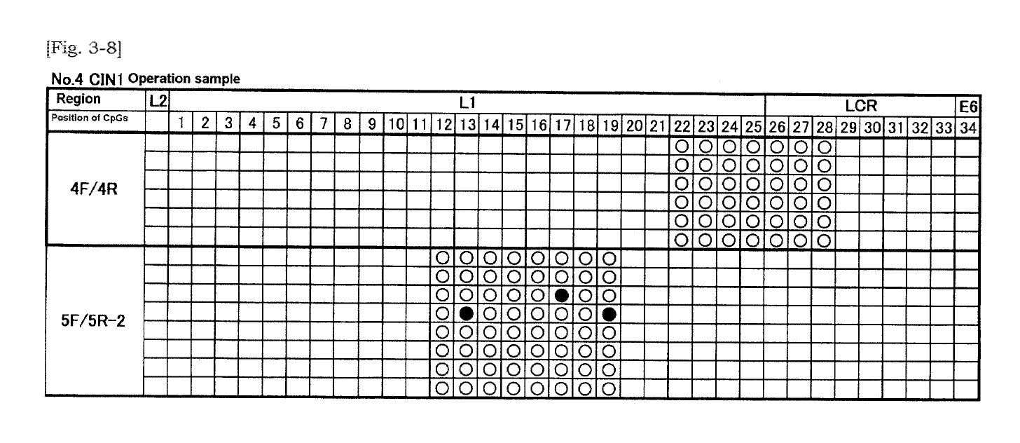

FIG. 3 shows the results of detection of methylated CpGs in L1 region or LCR of HPV-58 genomic DNA.

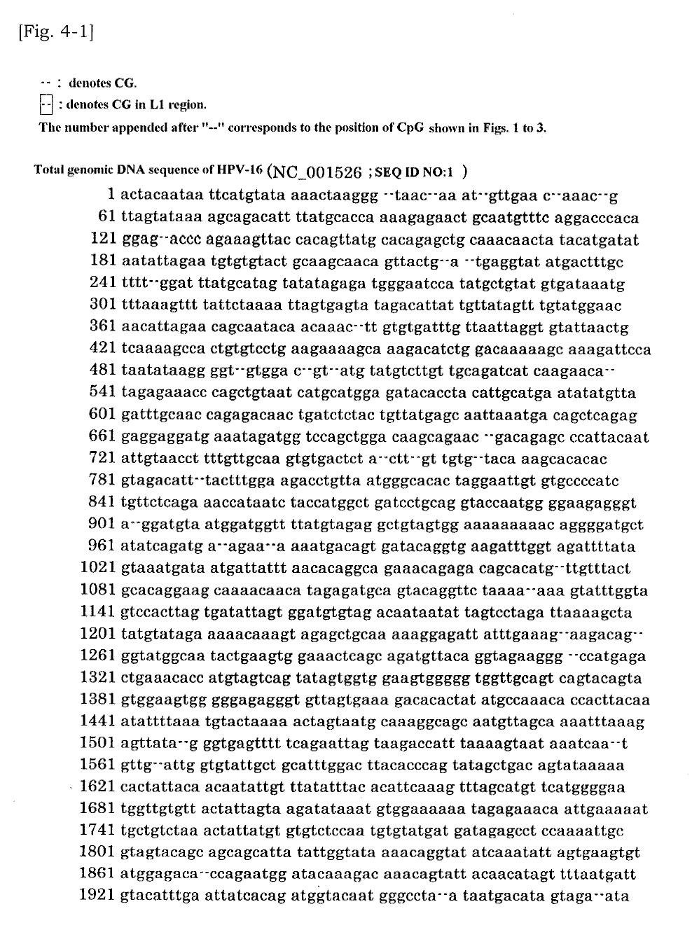

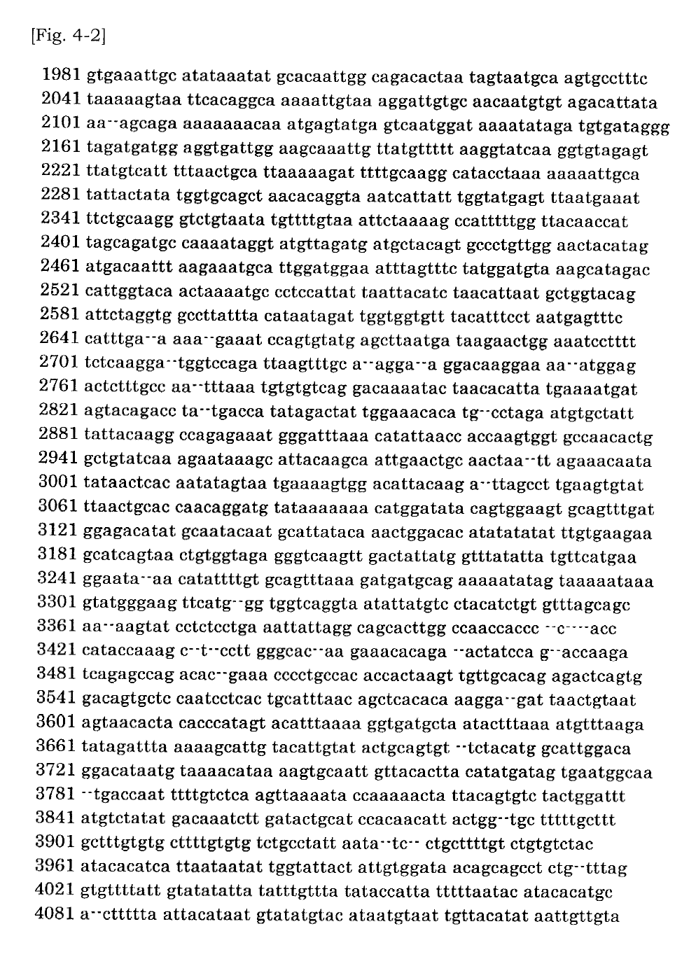

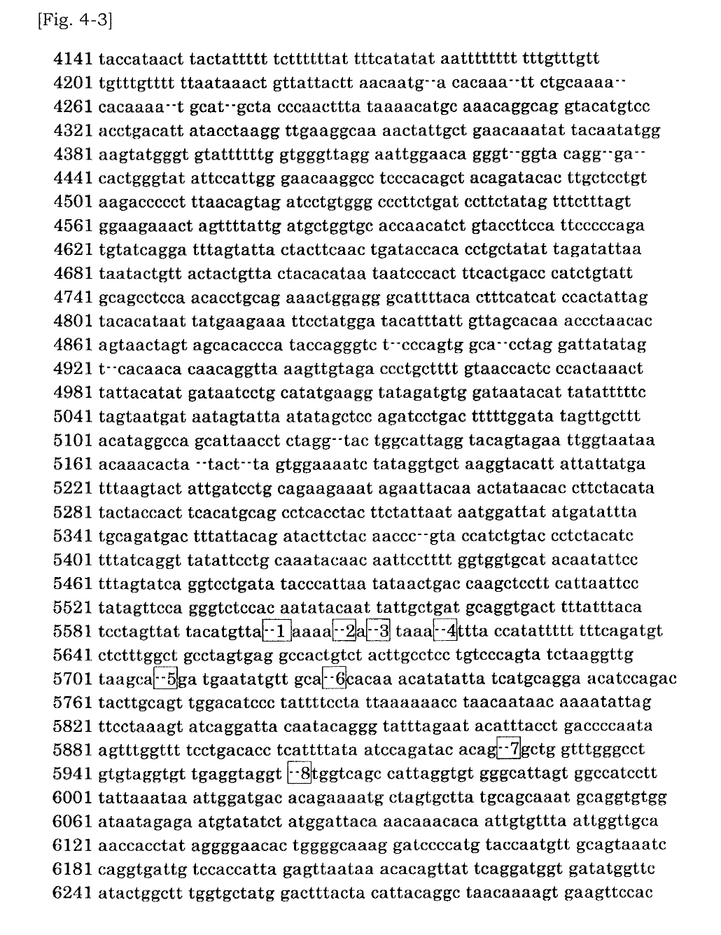

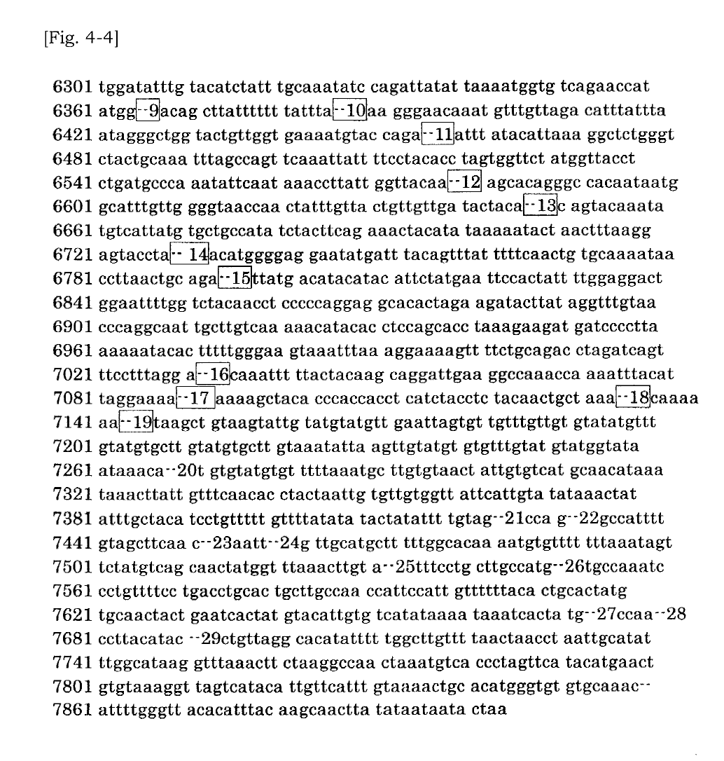

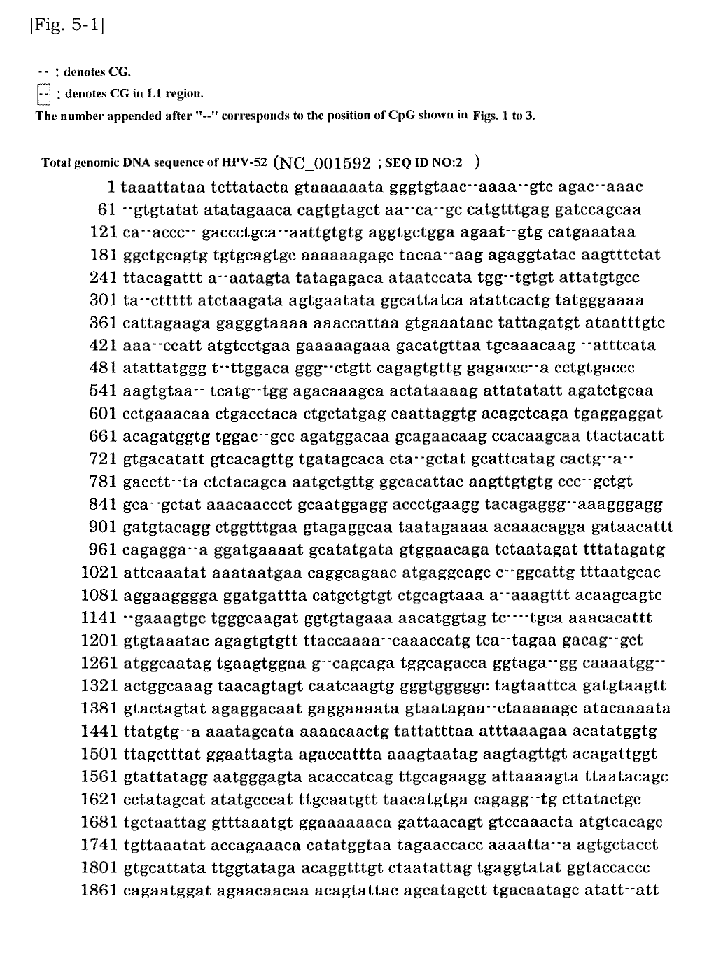

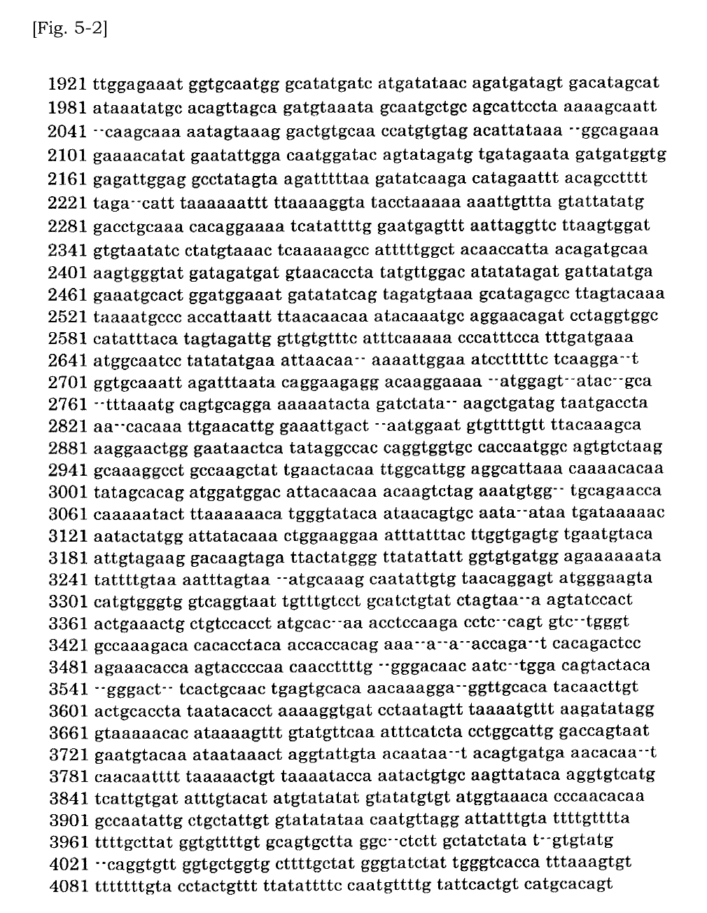

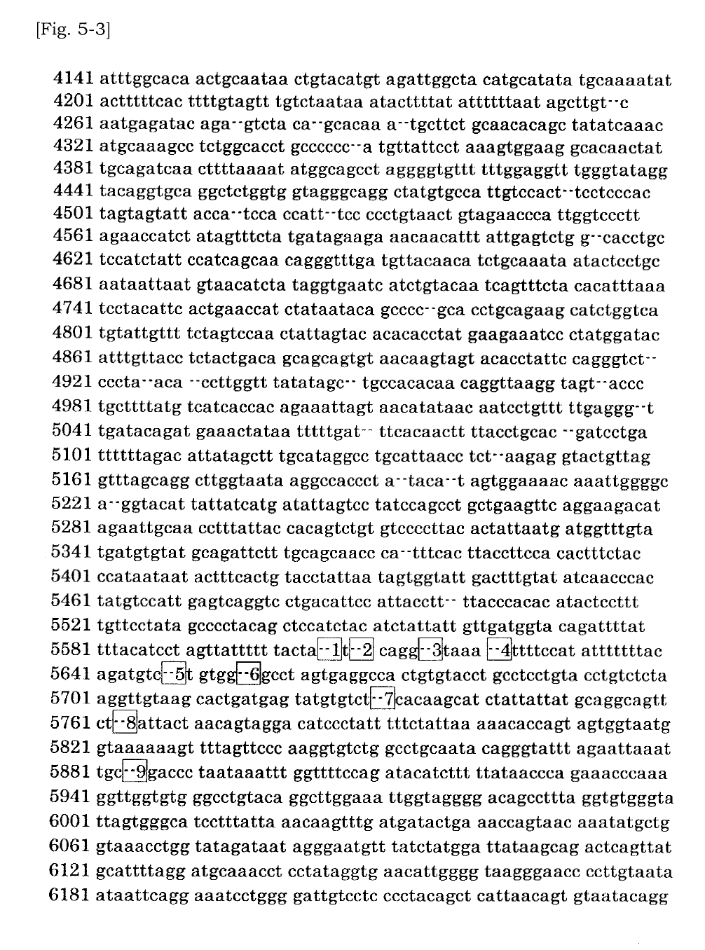

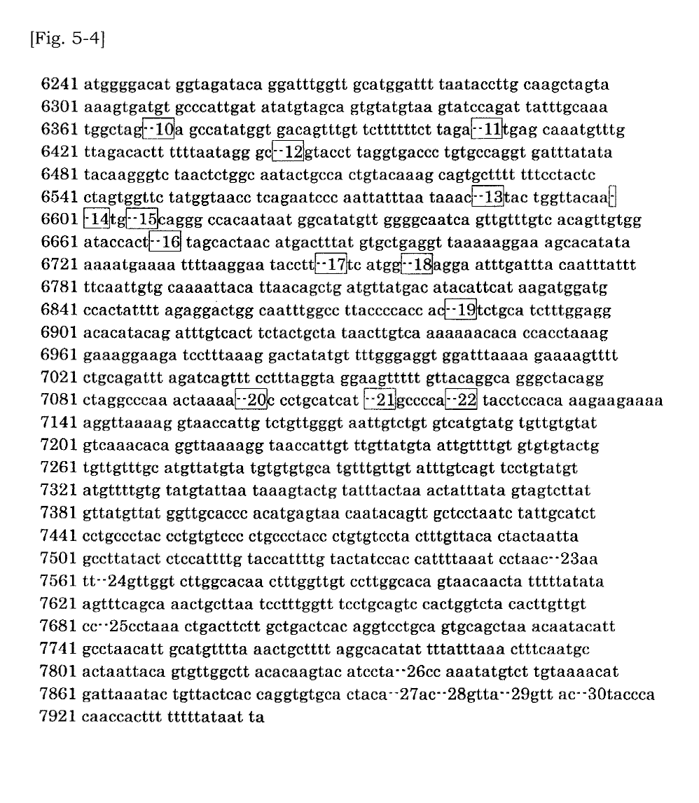

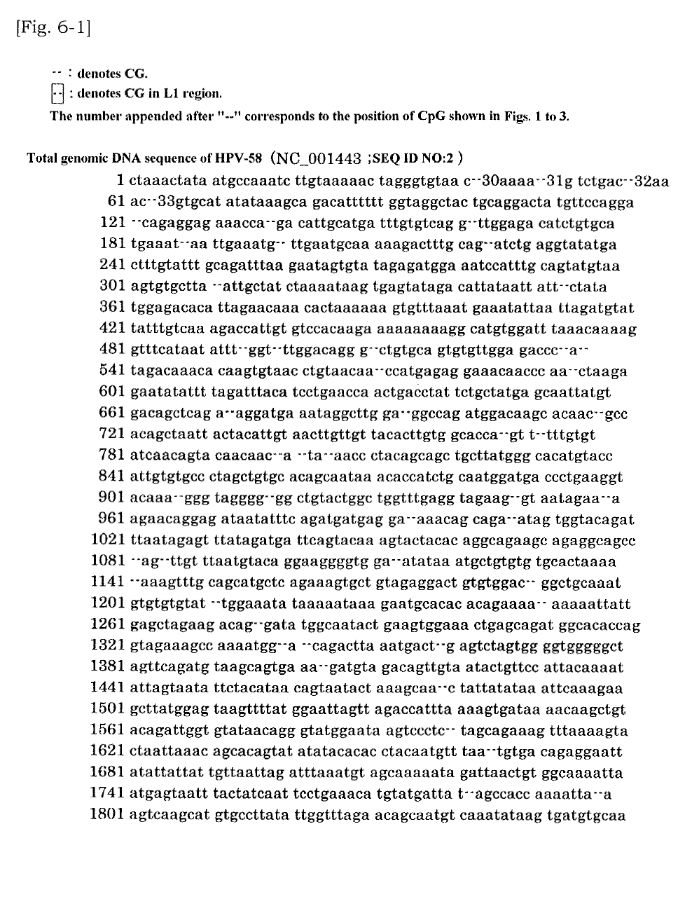

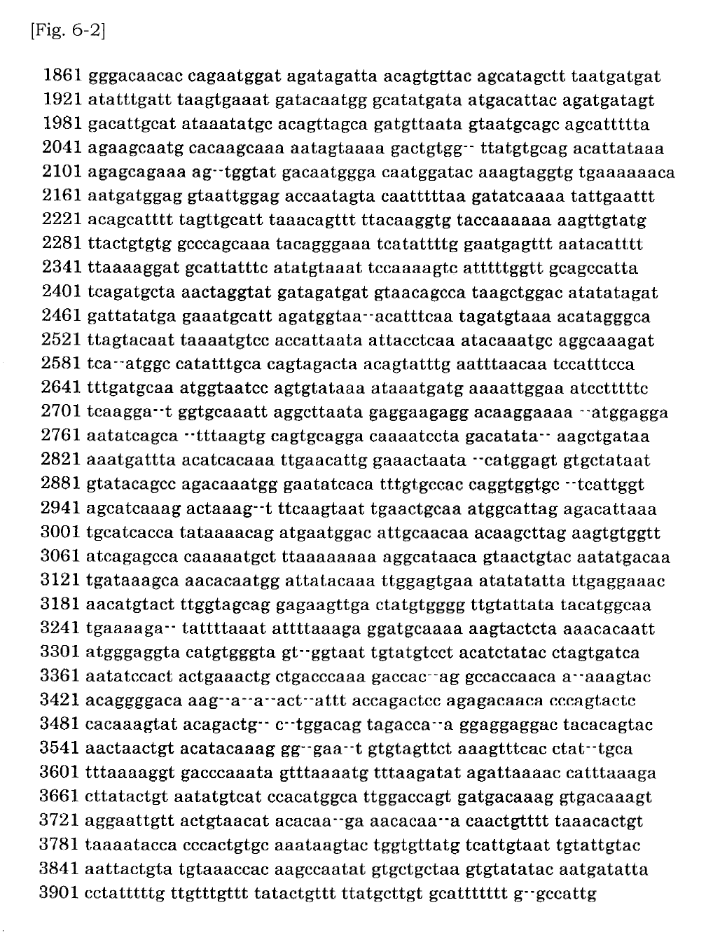

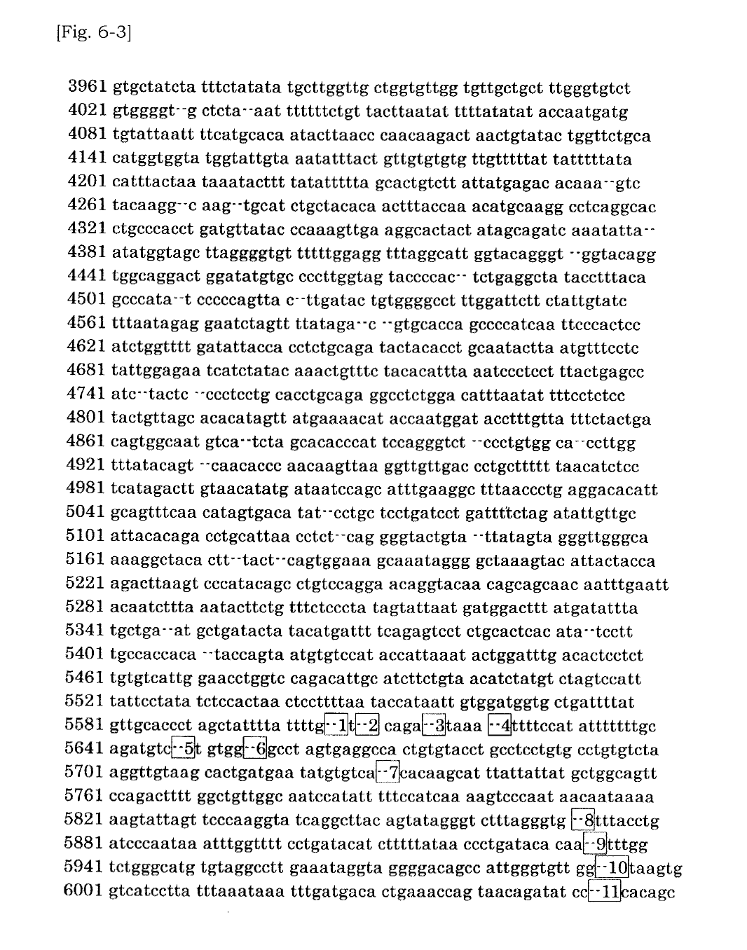

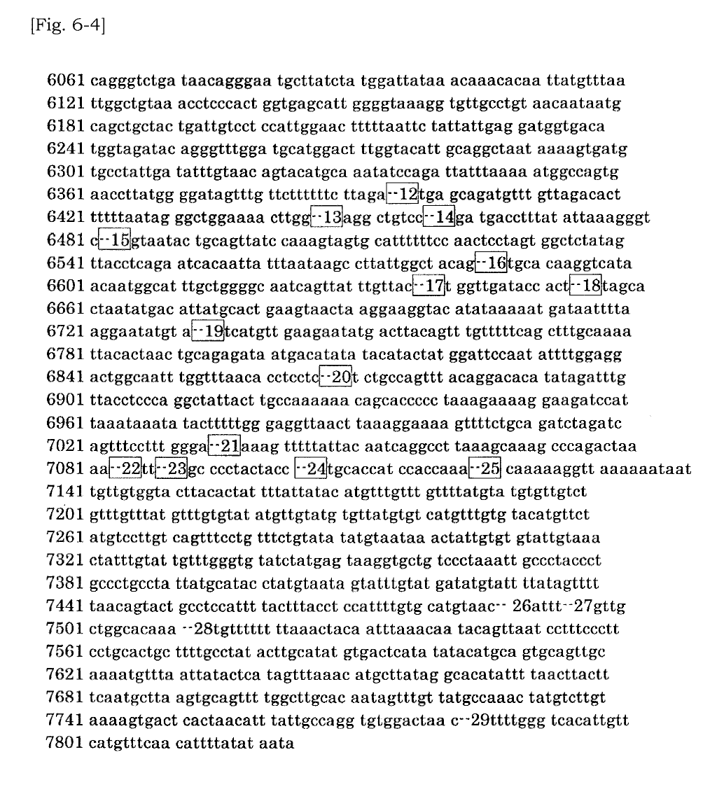

In FIGS. 1 to 3, the primer sets used for PCR following the bisulfite treatment are shown on the upper part of each Figure as, e.g. “6F/6R”. “Position of CpGs” in “L1” or “LCR” region corresponds to CpGs shown with numbers in FIGS. 4 to 6. For example, when the primer set “6F/6R” is used as shown in FIG. 1, the region comprising the 11^{th} to 15^{th} CpGs from 5′-terminal of L1 region of HPV-16 can be amplified and methylation of these CpGs can be measured.

In addition, FIGS. 1 to 3 show the results of the sequencing of several E. coli clones obtained by TA cloning of the products amplified by PCR with the primer sets. For example, for the subject No. 1 shown in FIG. 1, methylation was measured for seven E. coli clones containing the products amplified by PCR with the primer set “6F/6R”. Among CpGs in L1 region of HPV-16 measured for methylation, those which measured as “methylated” and “not methylated” are shown with and ∘, respectively.

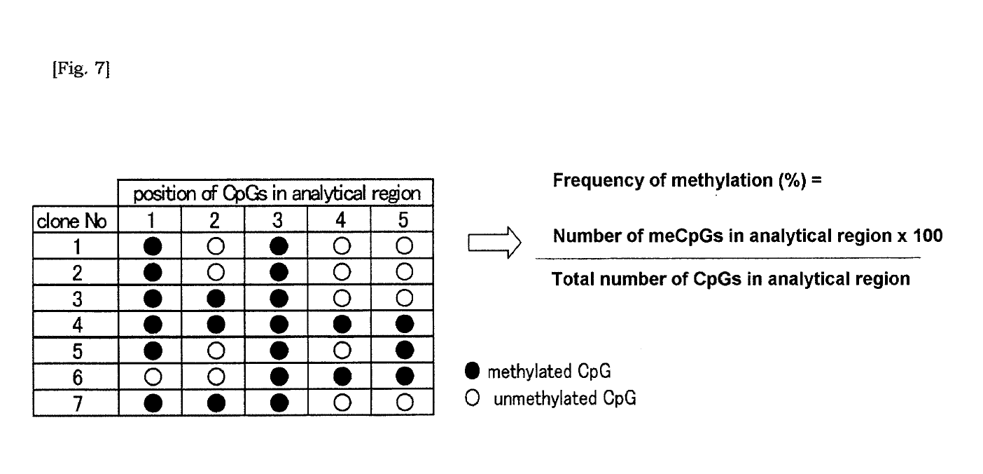

The frequency of methylation was calculated as shown in FIG. 7. Namely, the number of methylated CpG(s) among all CpGs in the analytical region was obtained from the results of all measured clones.

3-1. Results for Operation Samples and Discussion

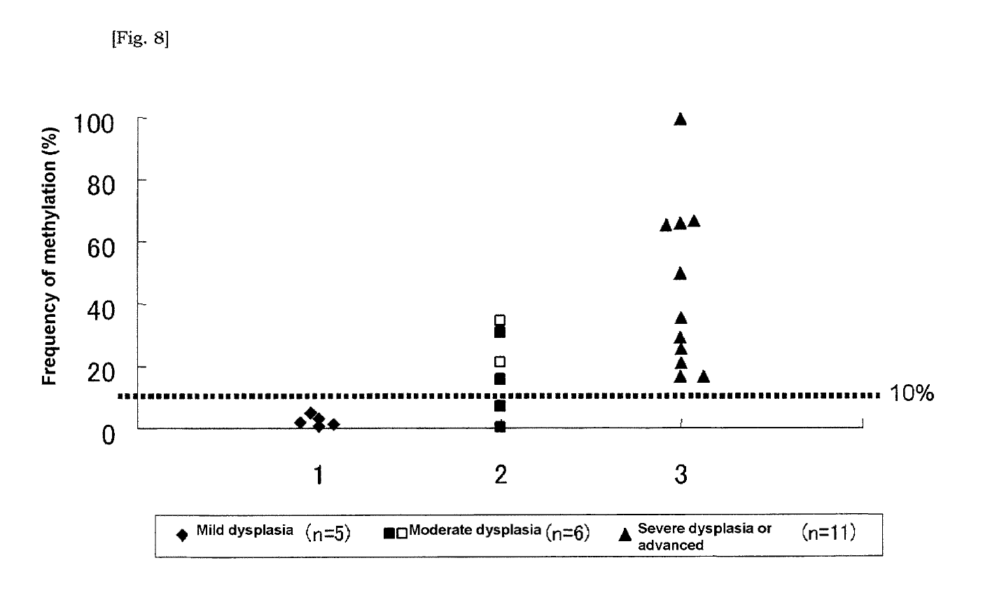

The frequencies of methylation in L1 regions of HPV-16, HPV-58 and HPV-52 obtained as above for the operation samples from total 22 subjects are shown in FIG. 8 as a scatter diagram in relation to the severity of lesion diagnosed by histological diagnosis.

Among 22 subjects diagnosed by histological diagnosis, 5 subjects were diagnosed as mild dysplasia (in the figure, shown as “1” and ♦), 6 were diagnosed as moderate dysplasia (in the figure, shown as “2” and ▪ and □) and 11 were diagnosed as severe dysplasia or lesion in more advanced stages (in the figure, shown as “3” and ▴). Among samples from the 6 subjects diagnosed as moderate dysplasia, the samples shown as □ were suspected as “severe dysplasia” by histological diagnosis.

The results in FIG. 8 shows that when the threshold is set to be the frequency of methylation of 10%, the samples from the subjects having severe dysplasia or lesion in more advanced stages can be clearly distinguished from those from other subjects. There were four samples which were diagnosed as moderate dysplasia by histological diagnosis and had the frequency of methylation of higher than 10%. Two samples among these were the samples suspected as “severe dysplasia” by histological diagnosis.

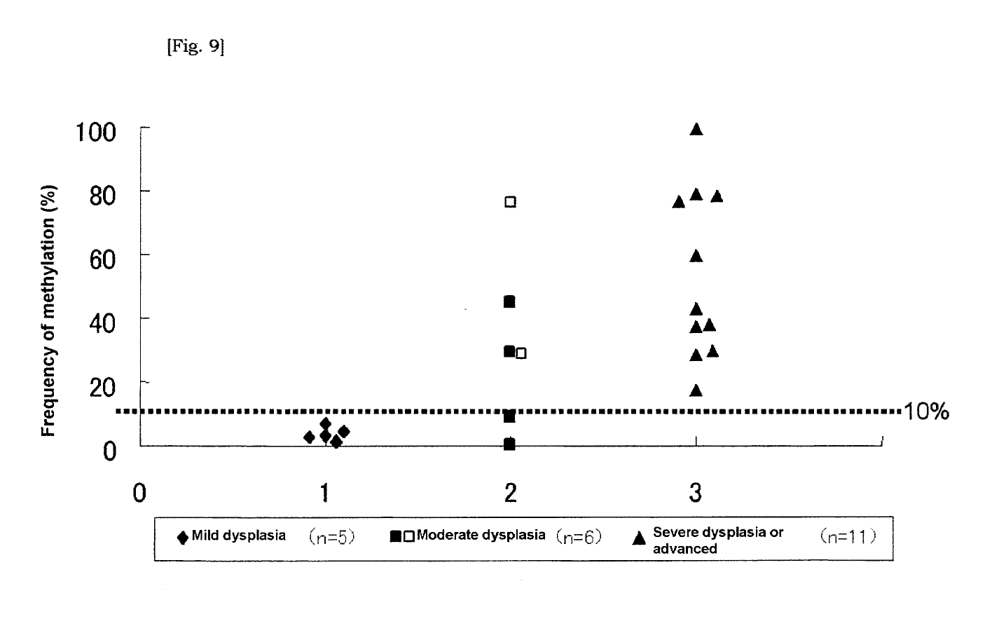

Among CpGs in L1 region, CpGs which exist within 80% from 5′-terminal were specifically analyzed and, similar to FIG. 8, a scatter diagram was obtained in FIG. 9 of the methylation frequency in relation to the severity of lesion diagnosed by histological diagnosis. CpGs which exist within 80% from 5′-terminal correspond to the 1^{st} to 15^{th} CpGs from 5′-terminal for HPV-16 (15/19=0.789). They correspond to the 1^{st} to 17^{th} CpGs (17/22=0.772) and the 1^{st} to 19^{th} CpGs (19/25=0.76) for HPV-52 and HPV-58, respectively.

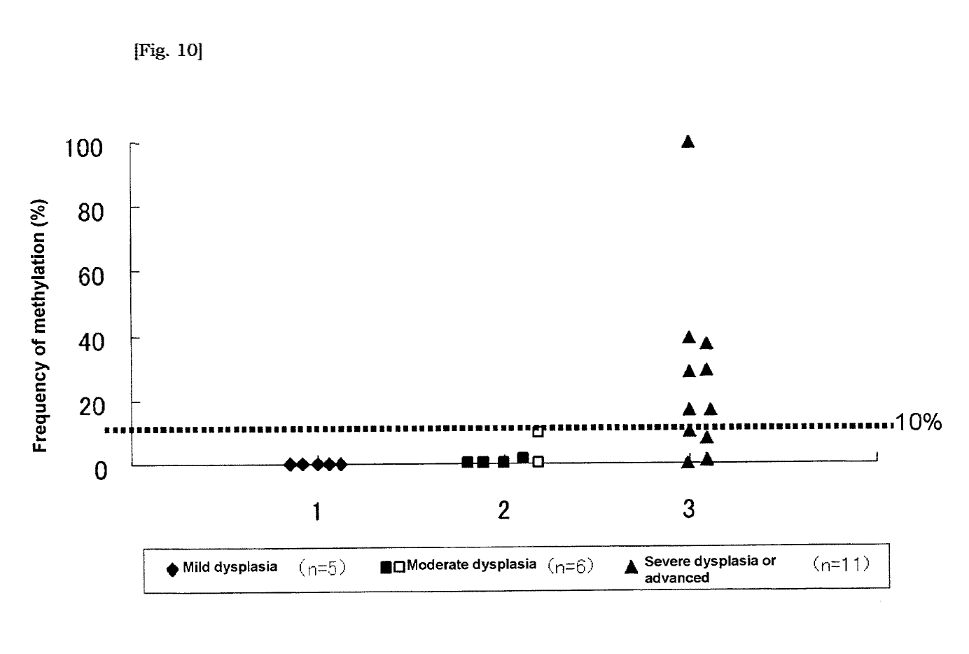

FIG. 10 shows a scatter diagram, as FIG. 8, of the frequency of methylation of the rest of CpGs, i.e. the 16^{th} to 19^{th}, the 18^{th} to 22^{nd} and the 20^{th} to 25^{th} CpGs from 5′-terminal for HPV-16, HPV-52 and HPV-58, respectively, in relation to the severity of lesion diagnosed by histological diagnosis.

The results in FIGS. 9 and 10 show that the results from the measurement of the frequency of methylation of CpGs existing within 80% from 5′-terminal among all CpGs in L1 region are in more conformity with the results of histological diagnosis than those from the measurement of the frequency of methylation of other CpGs.

3-2. Results for Biopsy Samples and Discussion

The frequencies of methylation in L1 regions of HPV-16, HPV-58 and HPV-52 were measured according to the present method for the biopsy samples obtained from 10 subjects, and whether they have severe dysplasia or lesion in more advanced stages () or not (∘) was detected with the threshold for the frequency of methylation of 10%.

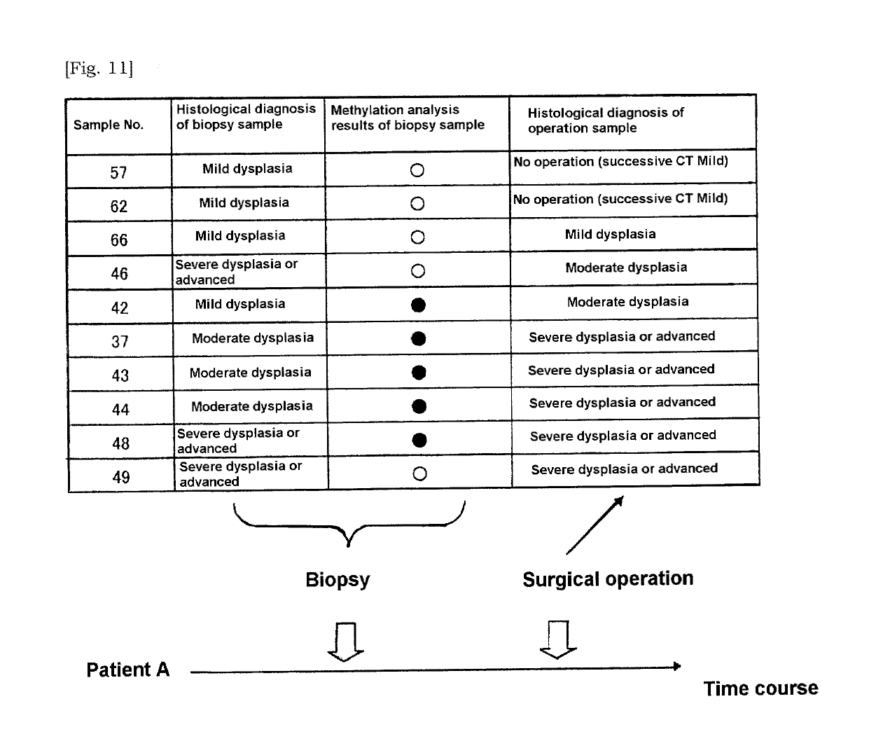

FIG. 11 shows the results of the determination according to the present method together with the results of histological diagnosis of the biopsy samples and results of histological diagnosis carried out on the operation samples taken from the same subjects.

Generally, histological diagnosis on biopsy samples is liable to give false diagnosis results due to the storage condition of the samples, difficulties in the sample preparation of tissue sections and the like. On the other hand, histological diagnosis on operation samples is likely to give more accurate diagnosis results than that on biopsy samples because the operation samples are tissue which contains higher number of cells.

The results in FIG. 11 show that the present method can give results which are rather in conformity with the results by histological diagnosis on the operation samples, even when the biopsy samples are analyzed.

Thus, it is found that the present method allows accurate determination on the presence or absence of abnormal cells originated from severe dysplasia or lesion in more advanced stages in samples, regardless of whether the samples are operation samples or biopsy samples.

### Example 2

The frequency of methylation in L1 region was measured on the paraffin blocks of the biopsy samples obtained at the first visit of seven subjects whose progressions have been followed and whose uterine cervical lesions have disappeared within three years from the first visit. Among these 7 subjects, two patients were infected with HPV-16, one with HPV-52 and four with HPV-58.

Similarly, the frequency of methylation in L1 region was measured on the paraffin blocks of the biopsy samples obtained at the first visit of nine subjects whose progressions have been followed and whose uterine cervical lesions have progressed to severe dysplasia or lesion in more advanced stages within three years from the first visit. Among these 9 subjects, two patients were infected with HPV-16, one with HPV-31, three with HPV-52 and three with HPV-58.

The frequency of methylation in L1 region was measured according to Example 1, except that the primer sets used were those shown in Table 3. The PCR conditions for the HPV-31 primer sets were the same as those for HPV-16/HPV-52.

TABLE 3

| | SEQ | | | | |
|---|---|---|---|---|---|
| Target | ID | | Amplified | Amplified | |
| HPV | NO: | Name | Region | size | Sequence |
| HPV-16 | 24 | 5F(16) | L1 | 439 bp | TTGTTGATGTAGGTGATTTTTATTTATATTTTAG |
| | | | | | TT |
| | 25 | 5R(16) | Anterior* | | CCACTAATACCCACACCTAATAACTAACC |
| | 32 | 5F-2(16) | L1 | 222 bp | GATGTAGGTGATTTTTATTTATATTTTAGTT |
| | 33 | 5R-2(16) | Anterior* | | ATCCAACTACAAATAATCTAAATATTC |
| | 6 | 7F(16) | L1-LCR | 440 bp | AGTAGGATTGAAGGTTAAATTAAAATTTAT |
| | 7 | 7R(16) | | | AACACATTTTATACCAAAAAACATACAACC |
| | 8 | 6F(16) | L1 | 424 bp | AATAGGGTTGGTATTGTTGGTGAAAAT |
| | 9 | 6R(16) | | | TTCCAATCCTCCAAAATAATAAAATTCATA |

| | | | | | |
|---|---|---|---|---|---|
| HPV-31 | 34 | 1F(31) | L1-LCR | 339 bp | GATTATGTATTTTGGGAGGTTAATTTAAAA |
| | 35 | 1R(31) | | | CATAACATACATACACACATAACATACTAT |
| | 36 | 3F(31) | L1 | 424 bp | GGTGATTGTTTTTTATTAGAATTAAAAA |
| | 37 | 3R(31) | | | AATACCATTATTATATCCCTAAACAC |
| HPV-52 | 26 | 5F(52) | L1 | 408 bp | TTTAGGTGATTTTGTGTTAGGT |
| | 27 | 5R(52) | Middle* | | TCCTCTAAAATAATAACATCCATCT |
| | 28 | 4F(52) | L1 | 385 bp | TTGGTTGTATGGATTTTAATATTT |
| | 29 | 4R(52) | Middle* | | AACAAACAACTAATTACCCCA |
| | 12 | 2F(52) | L1 | 367 bp | TTTATTTATATTTATTATTGTTGATGGTAT |
| | 13 | 2R(52) | Anterior* | | TAAAAAACCAAATTTATTAAAATCC |
| | 14 | 3F(52) | L1 | 299 bp | ATTTTAGAGGATTGGTAATTTGG |
| | 15 | 3R(52) | Posterior* | | AACCTTTTTCTTCTTTATAAAAATAC |
| HPV-58 | 30 | 5F(58) | L1 | 427 bp | TGGTTAGTGAATTTTATGGGG |
| | 31 | 5-2R(58) | Middle* | | TTACAAAACTAAAAAACAAACTATAAATCA |
| | 18 | 2F(58) | L1 | 415 bp | ATGGTGTTGATTTTATGTTGTATT |
| | 19 | 2R(58) | Anterior* | | AACTATCCCCTACCTATTTCAAAAC |
| | 20 | 3F(58) | L1 | 344 bp | TTAATATTTTGGAGGATTGGTAAT |
| | 21 | 3R(58) | Posterior* | | ATATAATAAAATAATATAAATACCACAACA |
| | 22 | 4F(58) | L1-LCR | 489 bp | AATTAGGTTTTAAAGTAAAGTTTAGATTA |
| | 23 | 4R(58) | | | TTATTTAAATTATAATTTAAAAAAAACAC |

*“L1 Anterior” means a region proximal to 5′-terminal of L1 region, “L1 Posterior” mans a region proximal to 3′-terminal of L1 region and “L1 Middle” means a central region of L1 region.

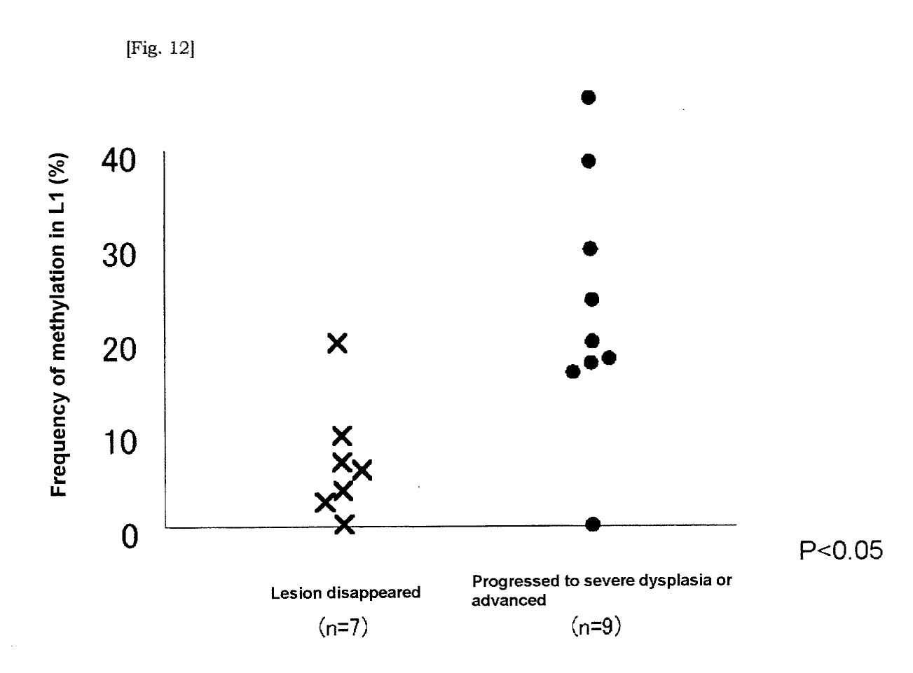

FIG. 12 shows a scatter diagram of the frequency of methylation (%) in L1 region at the first visit of the patients with different courses. According to FIG. 12, it is found that the frequencies of methylation at the first visit of the subjects whose lesion have progressed after the first visit to severe dysplasia or lesion in more advanced stages are higher than those of the subjects whose lesion have disappeared after the first visit.

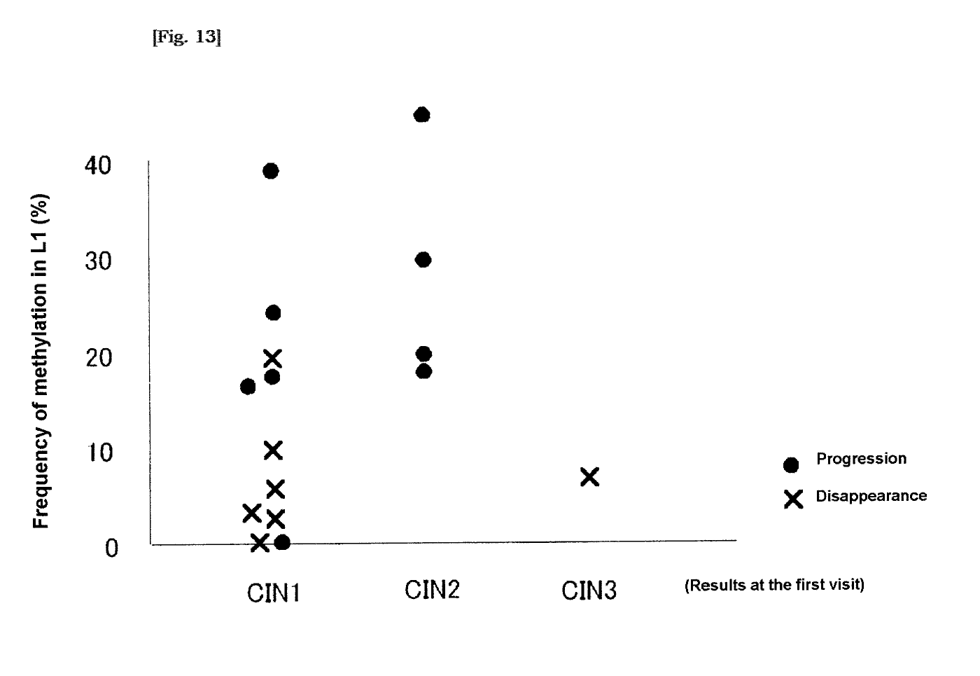

FIG. 13 shows a scatter diagram of the frequency of methylation in L1 region at the first visit of patients in relation to the severity of lesion diagnosed by histological diagnosis. In FIG. 13, “x” shows the subjects whose lesion have disappeared after the first visit and “” shows the subjects whose lesion have progressed to severe dysplasia or lesion in more advanced stages after the first visit. According to FIG. 13, it is found that when the frequency of methylation in L1 region is high, the lesion may progress with high possibility to severe dysplasia or lesion in more advanced stages even when it was diagnosed as mild dysplasia by histological diagnosis at the first visit. On the contrary, when the frequency of methylation in L1 region is low, the lesion may disappear after the first visit even when it was diagnosed as severe dysplasia or lesion in more advanced stages by histological diagnosis at the first visit.

These results indicate that the measurement of the frequency of methylation in L1 region of HPV genomic DNA in samples allows the prediction on whether or not uterine cervical tissue of subjects progresses to severe dysplasia or lesion in more advanced stages.

The present application relates to Japanese Patent Application No. 2008-273257 filed on Oct. 23, 2008, whose claims, specification, drawings and abstract are incorporated herein by reference.

1. A method of determining the presence or absence of abnormal cells originated from severe dysplasia or lesion in more advanced stages of uterine cervix in a sample obtained from uterine cervix of a subject, comprising the steps of:
measuring a frequency of methylation of 5′-(CG)-3′ (CpG) in L1 region of human papilloma virus genomic DNA in the sample; and
determining whether or not the abnormal cells are contained in the sample based on the measured frequency of methylation.

2. The method according to claim 1, wherein, in the step of determining, the measured frequency of methylation is compared to a predetermined threshold and the sample is determined to contain the abnormal cells when the frequency of methylation is higher than the threshold.

3. A method of predicting whether or not uterine cervix tissue of a subject progresses to severe dysplasia or lesion in more advanced stages, comprising the steps of:
measuring a frequency of methylation of 5′-(CG)-3′ (CpG) in L1 region of human papilloma virus genomic DNA in a sample obtained from uterine cervix of the subject; and
predicting whether or not the tissue progresses to severe dysplasia or lesion in more advanced stages based on the measured frequency of methylation.

4. The method according to claim 3, wherein, in the step of predicting, the measured frequency of methylation is compared to a predetermined threshold and the tissue is predicted to progress to severe dysplasia or lesion in more advanced stages when the frequency of methylation is higher than the threshold.

5. The method according to claim 1, wherein the frequency of methylation is obtained by dividing the number of methylated CpG(s) present in said L1 region which is subjected to the measurement of the frequency of methylation by the number of all CpGs present in said L1 region.

6. The method according to claim 1, wherein said L1 region which is subjected to the measurement of the frequency of methylation is a region which comprises at least one CpG existing within 80% from 5′-terminal among all CpGs in the L1 region.

7. The method according to claim 1, wherein human papilloma virus is at least one selected from HPV-16, HPV-18, HPV31, HPV33, HPV35, HPV-52 and HPV-58.

8. The method according to claim 7, wherein human papilloma virus is HPV-16 and wherein said L1 region which is subjected to the measurement of the frequency of methylation is a region which comprises at least one CpG among the 1^{st} to 15^{th} CpGs from 5′-terminal of L1 region of HPV-16 and does not comprise the 16^{th} to 19^{th} CpGs.

9. The method according to claim 7, wherein human papilloma virus is HPV-31 and wherein said L1 region which is subjected to the measurement of the frequency of methylation is a region which comprises at least one CpG among the 1^{st} to 17^{th} CpGs from 5′-terminal of L1 region of HPV-31 and does not comprise the 18^{th} to 22^{nd} CpGs.

10. The method according to claim 7, wherein human papilloma virus is HPV-52 and wherein said L1 region which is subjected to the measurement of the frequency of methylation is a region which comprises at least one CpG among the 1^{st} to 17^{th} CpGs from 5′-terminal of L1 region of HPV-52 and does not comprise the 18^{th} to 22^{nd} CpGs.

11. The method according to claim 7, wherein human papilloma virus is HPV-58 and wherein said L1 region which is subjected to the measurement of the frequency of methylation is a region which comprises at least one CpG among the 1^{st} to 19^{th} CpGs from 5′-terminal of L1 region of HPV-58 and does not comprise the 20^{th} to 25^{th} CpGs.

12. The method according to claim 1, wherein severe dysplasia or lesion in more advanced stages includes severe dysplasia, intraepithelial cancer, microinvasive squamous cancer and invasive squamous cancer.

13. A primer set for determining the presence or absence of abnormal cells originated from severe dysplasia or lesion in more advanced stages of uterine cervix, or for predicting the progress to severe dysplasia or lesion in more advanced stages, which is used in a nucleic acid amplification method for amplification of a region comprising at least one CpG existing within 80% from 5′-terminal among all CpGs in L1 region of human papilloma virus genomic DNA, said region having been treated with bisulfite.

14. The primer set according to claim 13, wherein HPV is at least one selected from HPV-16, HPV-18, HPV-31, HPV33, HPV35, HPV-52 and HPV-58.

15. The primer set according to claim 14, wherein HPV is HPV-16 and wherein the region amplified in the amplification method is a region which comprises at least one CpG among the 1^{st} to 15^{th} CpGs from 5′-terminal of L1 region of HPV-16 and does not comprise the 16^{th} to 19^{th} CpGs, said region having been treated with bisulfite.

16. The primer set according to claim 14, wherein HPV is HPV-18 and wherein the region amplified in the amplification method is a region which comprises at least one CpG among the 1^{st} to 25^{th} CpGs from 5′-terminal of L1 region of HPV-18 and does not comprise the 26^{th} to 32^{nd} CpGs, said region having been treated with bisulfite.

17. The primer set according to claim 14, wherein HPV is HPV-31 and wherein the region amplified in the amplification method is a region which comprises at least one CpG among the 1^{st} to 17^{th} CpGs from 5′-terminal of L1 region of HPV-31 and does not comprise the 18^{th} to 22^{nd} CpGs, said region having been treated with bisulfite.

18. The primer set according to claim 14, wherein HPV is HPV-33 and wherein the region amplified in the amplification method is a region which comprises at least one CpG among the 1^{st} to 16^{th} CpGs from 5′-terminal of L1 region of HPV-33 and does not comprise the 17^{th} to 21^{st} CpGs, said region having been treated with bisulfite.

19. The primer set according to claim 14, wherein HPV is HPV-35 and wherein the region amplified in the amplification method is a region which comprises at least one CpG among the 1^{st} to 13^{th} CpGs from 5′-terminal of L1 region of HPV-35 and does not comprise the 14^{th} to 17^{th} CpGs, said region having been treated with bisulfite.

20. The primer set according to claim 14, wherein HPV is HPV-52 and wherein the region amplified in the amplification method is a region which comprises at least one CpG among the 1^{st} to 17^{th} CpGs from 5′-terminal of L1 region of HPV-52 and does not comprise the 18^{th} to 22^{nd} CpGs, said region having been treated with bisulfite.

21. The primer set according to claim 14, wherein HPV is HPV-58 and wherein the region amplified in the amplification method is a region which comprises at least one CpG among the 1^{st} to 19^{th} CpGs from 5′-terminal of L1 region of HPV-58 and does not comprise the 20^{th} to 25^{th} CpGs, said region having been treated with bisulfite.
